SEQUENCE LISTING (66 sequences)

SEQ ID NO: 1 - DNA, 20 bp, Artificial Sequence
  source: /note=“Description of Artificial Sequence Synthetic primer”
gtggtggtacgggaaatcac

SEQ ID NO: 2 - DNA, 20 bp, Artificial Sequence
  source: /note=“Description of Artificial Sequence Synthetic primer”
tagccaggttcgagaatcca

SEQ ID NO: 3 - DNA, 20 bp, Artificial Sequence
  source: /note=“Description of Artificial Sequence Synthetic primer”
gcatcgcagcttggatacac

SEQ ID NO: 4 - DNA, 19 bp, Artificial Sequence
  source: /note=“Description of Artificial Sequence Synthetic primer”
gcttcagctccgtctccat

SEQ ID NO: 5 - DNA, 20 bp, Artificial Sequence
  source: /note=“Description of Artificial Sequence Synthetic primer”
ctgggttgatcctcggacct

SEQ ID NO: 6 - DNA, 20 bp, Artificial Sequence
  source: /note=“Description of Artificial Sequence Synthetic primer”
cacagaactcatacggcggg

SEQ ID NO: 7 - DNA, 20 bp, Artificial Sequence
  source: /note=“Description of Artificial Sequence Synthetic primer”
aaagaatcttcacctatgcc

SEQ ID NO: 8 - DNA, 20 bp, Artificial Sequence
  source: /note=“Description of Artificial Sequence Synthetic primer”
gaaggaagaggagagacagt

SEQ ID NO: 9 - DNA, 20 bp, Artificial Sequence
  source: /note=“Description of Artificial Sequence Synthetic primer”
tccttctacggacggaactg

SEQ ID NO: 10 - DNA, 20 bp, Artificial Sequence
  source: /note=“Description of Artificial Sequence Synthetic primer”
agaaatgcctgaggaaagca

SEQ ID NO: 11 - DNA, 20 bp, Artificial Sequence
  source: /note=“Description of Artificial Sequence Synthetic primer”
cacaactcggagatcagcaa

SEQ ID NO: 12 - DNA, 20 bp, Artificial Sequence
  source: /note=“Description of Artificial Sequence Synthetic primer”
ggtacttgtaatccgggtgc

SEQ ID NO: 13 - DNA, 20 bp, Artificial Sequence
  source: /note=“Description of Artificial Sequence Synthetic primer”
gtccatctttgcttgggaaa

SEQ ID NO: 14 - DNA, 20 bp, Artificial Sequence
  source: /note=“Description of Artificial Sequence Synthetic primer”
tagccaggttgcgaagaact

SEQ ID NO: 15 - DNA, 30 bp, Artificial Sequence
  source: /note=“Description of Artificial Sequence Synthetic primer”
caggtggcggacgtgtgaaaattgagagtg

SEQ ID NO: 16 - DNA, 26 bp, Artificial Sequence
  source: /note=“Description of Artificial Sequence Synthetic primer”
cacgctggatctgcctggggactgtg

SEQ ID NO: 17 - DNA, 20 bp, Artificial Sequence
  source: /note=“Description of Artificial Sequence Synthetic primer”
aattggtccagccttggaat

SEQ ID NO: 18 - DNA, 18 bp, Artificial Sequence
  source: /note=“Description of Artificial Sequence Synthetic primer”
cgttgctcacagaccaca

SEQ ID NO: 19 - DNA, 20 bp, Artificial Sequence
  source: /note=“Description of Artificial Sequence Synthetic primer”
tgatcggaaatgacactgga

SEQ ID NO: 20 - DNA, 20 bp, Artificial Sequence
  source: /note=“Description of Artificial Sequence Synthetic primer”
cacgactccatgttggtcac

SEQ ID NO: 21 - DNA, 24 bp, Artificial Sequence
  source: /note=“Description of Artificial Sequence Synthetic primer”
cgagaggaccccgtggatgcagag

SEQ ID NO: 22 - DNA, 24 bp, Artificial Sequence
  source: /note=“Description of Artificial Sequence Synthetic primer”
ggcggccatcttcagcttctccag

SEQ ID NO: 23 - DNA, 18 bp, Artificial Sequence
  source: /note=“Description of Artificial Sequence Synthetic primer”
ctctgcctcctccacgaa

SEQ ID NO: 24 - DNA, 20 bp, Artificial Sequence
  source: /note=“Description of Artificial Sequence Synthetic primer”
cagaatccagacctgcacaa

SEQ ID NO: 25 - DNA, 18 bp, Artificial Sequence
  source: /note=“Description of Artificial Sequence Synthetic primer”
ggagcggtgaagatggaa

SEQ ID NO: 26 - DNA, 20 bp, Artificial Sequence
  source: /note=“Description of Artificial Sequence Synthetic primer”
tacgtgttcatgccgttcat

SEQ ID NO: 27 - DNA, 20 bp, Artificial Sequence
  source: /note=“Description of Artificial Sequence Synthetic primer”
aaatgcgtttctcgttgctt

SEQ ID NO: 28 - DNA, 20 bp, Artificial Sequence
  source: /note=“Description of Artificial Sequence Synthetic primer”
gccacaggccaatagtttgt

SEQ ID NO: 29 - DNA, 26 bp, Artificial Sequence
  source: /note=“Description of Artificial Sequence Synthetic primer”
cgccaggatccccgctttgaaatctg

SEQ ID NO: 30 - DNA, 26 bp, Artificial Sequence
  source: /note=“Description of Artificial Sequence Synthetic primer”
tcggccgccagctctttgatgtgttc

SEQ ID NO: 31 - DNA, 26 bp, Artificial Sequence
  source: /note=“Description of Artificial Sequence Synthetic primer”
acagaggggaggtgcgccagttcacg

SEQ ID NO: 32 - DNA, 26 bp, Artificial Sequence
  source: /note=“Description of Artificial Sequence Synthetic primer”
acggggtggacctcgctgcacagatc

SEQ ID NO: 33 - DNA, 24 bp, Artificial Sequence
  source: /note=“Description of Artificial Sequence Synthetic primer”
ggaggcgtggagctcagcgacacc

SEQ ID NO: 34 - DNA, 24 bp, Artificial Sequence
  source: /note=“Description of Artificial Sequence Synthetic primer”
cggggagctcgctgacggagtctg

SEQ ID NO: 35 - DNA, 26 bp, Artificial Sequence
  source: /note=“Description of Artificial Sequence Synthetic primer”
ggcaccagcagcagcaggagcagcag

SEQ ID NO: 36 - DNA, 26 bp, Artificial Sequence
  source: /note=“Description of Artificial Sequence Synthetic primer”
ccacgtctgaggagccgaggaagcag

SEQ ID NO: 37 - DNA, 20 bp, Artificial Sequence
  source: /note=“Description of Artificial Sequence Synthetic primer”
ctgtggcctttgaggagaag

SEQ ID NO: 38 - DNA, 20 bp, Artificial Sequence
  source: /note=“Description of Artificial Sequence Synthetic primer”
atggtggattttggcttcaa

SEQ ID NO: 39 - DNA, 20 bp, Artificial Sequence
  source: /note=“Description of Artificial Sequence Synthetic primer”
ctcccaacatgaccctgagt

SEQ ID NO: 40 - DNA, 20 bp, Artificial Sequence
  source: /note=“Description of Artificial Sequence Synthetic primer”
ctcattgcacgctgcataat

SEQ ID NO: 41 - DNA, 21 bp, Artificial Sequence
  source: /note=“Description of Artificial Sequence Synthetic primer”
cagagcggaaaagtgggaaga

SEQ ID NO: 42 - DNA, 21 bp, Artificial Sequence
  source: /note=“Description of Artificial Sequence Synthetic primer”
tcgttgatcctgtttcggaga

SEQ ID NO: 43 - DNA, 28 bp, Artificial Sequence
  source: /note=“Description of Artificial Sequence Synthetic primer”
tttaacaccgccagcgctcttcaccttg

SEQ ID NO: 44 - DNA, 28 bp, Artificial Sequence
  source: /note=“Description of Artificial Sequence Synthetic primer”
tcgtggcgcgtgtgttgtgggtatctcg

SEQ ID NO: 45 - DNA, 26 bp, Artificial Sequence
  source: /note=“Description of Artificial Sequence Synthetic primer”
gggccccatcaacttcaccgtcttcc

SEQ ID NO: 46 - DNA, 26 bp, Artificial Sequence
  source: /note=“Description of Artificial Sequence Synthetic primer”
tgtagtcgatgttccccgccaggtcc

SEQ ID NO: 47 - DNA, 25 bp, Artificial Sequence
  source: /note=“Description of Artificial Sequence Synthetic primer”
ctggaggccgagcagaagcgcaacg

SEQ ID NO: 48 - DNA, 25 bp, Artificial Sequence
  source: /note=“Description of Artificial Sequence Synthetic primer”
gtccgcccgctcctctgcctcatcc

SEQ ID NO: 49 - DNA, 20 bp, Artificial Sequence
  source: /note=“Description of Artificial Sequence Synthetic primer”
cgagcagatccagttcatca

SEQ ID NO: 50 - DNA, 20 bp, Artificial Sequence
  source: /note=“Description of Artificial Sequence Synthetic primer”
cgttggttcccatatgttcc

SEQ ID NO: 51 - DNA, 20 bp, Artificial Sequence
  source: /note=“Description of Artificial Sequence Synthetic primer”
cctttcccatggatgaagtc

SEQ ID NO: 52 - DNA, 21 bp, Artificial Sequence
  source: /note=“Description of Artificial Sequence Synthetic primer”
ggaactccttctccagctcta

SEQ ID NO: 53 - DNA, 20 bp, Artificial Sequence
  source: /note=“Description of Artificial Sequence Synthetic primer”
gtggacctgacctgccgtct

SEQ ID NO: 54 - DNA, 20 bp, Artificial Sequence
  source: /note=“Description of Artificial Sequence Synthetic primer”
ggaggagtgggtgtcgctgt

SEQ ID NO: 55 - DNA, 20 bp, Artificial Sequence
  source: /note=“Description of Artificial Sequence Synthetic primer”
caatgtggccgaggactttg

SEQ ID NO: 56 - DNA, 22 bp, Artificial Sequence
  source: /note=“Description of Artificial Sequence Synthetic primer”
cattctccttagagagaagtgg

SEQ ID NO: 57 - DNA, 20 bp, Artificial Sequence
  source: /note=“Description of Artificial Sequence Synthetic primer”
gcatcgcagcttggatacac

SEQ ID NO: 58 - DNA, 20 bp, Artificial Sequence
  source: /note=“Description of Artificial Sequence Synthetic primer”
ccaaggtgatcctcttctgc

SEQ ID NO: 59 - DNA, 20 bp, Artificial Sequence
  source: /note=“Description of Artificial Sequence Synthetic primer”
gcatcgcagcttggatacac

SEQ ID NO: 60 - DNA, 19 bp, Artificial Sequence
  source: /note=“Description of Artificial Sequence Synthetic primer”
gcttcagctccgtctccat

SEQ ID NO: 61 - DNA, 20 bp, Artificial Sequence
  source: /note=“Description of Artificial Sequence Synthetic primer”
gcatcgcagcttggatacac

SEQ ID NO: 62 - DNA, 20 bp, Artificial Sequence
  source: /note=“Description of Artificial Sequence Synthetic primer”
aaggccctgtcacacttctg

SEQ ID NO: 63 - DNA, 27 bp, Artificial Sequence
  source: /note=“Description of Artificial Sequence Synthetic primer”
ggatgttattaagatgaagatagttgg

SEQ ID NO: 64 - DNA, 25 bp, Artificial Sequence
  source: /note=“Description of Artificial Sequence Synthetic primer”
cctaaactccccttcaaaatctatt

SEQ ID NO: 65 - DNA, 25 bp, Artificial Sequence
  source: /note=“Description of Artificial Sequence Synthetic primer”
tagttgggatgtgtagagtttgaga

SEQ ID NO: 66 - DNA, 25 bp, Artificial Sequence
  source: /note=“Description of Artificial Sequence Synthetic primer”
taaaccaaaacaatccttctactcc

(19) United States
(12) Patent Application Publication
(10) Pub. No.: US 2011/0014164 A1
(43) Pub. Date: Jan. 20, 2011

(54) EFFICIENT INDUCTION OF PLURIPOTENT STEM CELLS USING SMALL MOLECULE COMPOUNDS

(75) Inventors: Danwei Huangfu, Belmont, MA (US); Douglas A. Melton, Lexington, MA (US); Rene Maehr, Somerville, MA (US)

Correspondence Address: DAVID S. RESNICK, NIXON PEABODY LLP, 100 SUMMER STREET, BOSTON, MA, 02110-2131, US

(73) Assignee: PRESIDENT AND FELLOWS OF HARVARD COLLEGE, Cambridge, MA (US)

(21) Appl. No.: 12/867,615

(22) PCT Filed: Feb. 13, 2009
(86) PCT No.: PCT/US09/34102

§ 371 Date: Sep. 22, 2010

Related U.S. Application Data
(60) Provisional application No. 61/029,287, filed on Feb. 15, 2008.
(60) Provisional application No. 61/091,004, filed on Aug. 22, 2008.

(51) Int. Cl. A61K 35/12; C12N 5/07; C12N 5/0797; C12N 5/071; G01N 33/566; A61P 25/28; A61P 17/02; A61P 9/00; A61P 9/10; A61P 1/16; A61P 3/10; A61P 5/14; A61P 11/00; A61P 19/08; A61P 35/02; A61P 37/02; A61P 7/06

(52) U.S. Cl. 424/93.7; 435/377; 435/366; 435/7.21

(57) ABSTRACT

The disclosure features a method of producing an induced pluripotent stem cell a somatic cell. The method includes contacting a somatic cell with a DNA methyl transferase inhibitor or a histone deacetylase (HDAC) inhibitor, or a combination thereof, to produce a pluripotent stem cell.

### BACKGROUND

The invention relates to the conversion of a somatic cell into more a primitive precursor, e.g., stem cell such as an induced pluripotent stem cell.

### SUMMARY

The methods described herein can be used, for example, to optimize (e.g., improve speed or efficiency) the creation of induced cells, e.g., induced pluripotent stem (iPS) cells from other cell types (e.g., an adult cell and/or a somatic cell), including, but not limited to the creation of iPS cells from human biopsies, such as blood, skin, fat, hair follicle, mucus, etc. The iPS lines so created can be used to study differentiation and disease mechanisms/pathology.

In one aspect, the invention features a method of producing an iPS cell from a somatic cell, the method comprising: treating the somatic cell with at least two transcription factors and contacting the somatic cell with a DNA methyl transferase inhibitor or a histone deacetylase (HDAC) inhibitor under conditions sufficient to produce an iPS cell from the somatic cell.

In some embodiments, the somatic cell is treated with at least two transcription factors prior to the step of contacting the somatic cell with the DNA methyl transferase inhibitor or the HDAC inhibitor.

In some embodiments, the step of treating the somatic cell with at least two transcription factors comprises treating the somatic cell with at least one heterologous nucleic acid sequence encoding at least two transcription factors.

In some embodiments, the somatic cell is treated with at least one heterologous nucleic acid sequence encoding at least two transcription factors by infection.

In some embodiments, the DNA methyl transferase inhibitor comprises 5′-azacytidine.

In some embodiments, the HDAC inhibitor selectively inhibits a Class I or Class II HDAC.

In some embodiments, the HDAC inhibitor comprises VPA, SAHA or TSA, or a combination thereof.

In some embodiments, the HDAC inhibitor comprises VPA.

In some embodiments, the method further comprises the step of contacting the cell with a glucocorticoid compound.

In some embodiments, the glucocorticoid compound comprises dexamethasone.

In some embodiments, the transcription factors comprise Oct4, Klf4, Sox2 or c-Myc.

In some embodiments, the method comprises treating the somatic cell with two transcription factors.

In some embodiments, the transcription factors comprise Oct4 and Sox2.

In some embodiments, the method comprises treating the somatic cell with three transcription factors.

In some embodiments, the transcription factors comprise Oct4, Sox2 and Klf4.

In some embodiments, the method comprises treating the somatic cell with four transcription factors.

In some embodiments, the transcription factors comprise Oct4, Sox2, Klf4 and c-Myc.

In some embodiments, the expression of a marker selected from a group consisting of alkaline phophatase, NANOG, OCT4, SOX2, SSEA4, TRA-1-60 and TRA-1-81, is upregulated to by a statistically significant amount in the iPS cell relative to the somatic cell.

In some embodiments, the iPS cell has a normal karyotype.

In some embodiments, the somatic cell is a fibroblast (e.g., a primary fibroblast), a muscle cell (e.g., a myocyte), a cumulus cell, a neural cell, a liver cell, a GI tract cell, a mammary cell, a hepatocyte or a pancreatic islet cell.

In some embodiments, the somatic cell is a primary cell or is a progeny of a primary or secondary cell.

In some embodiments, the somatic cell is a human cell.

In some embodiments, the somatic cell is obtained from a sample selected from a group consisting of a hair follicle, a blood sample, a swab sample or an adipose biopsy.

In some embodiments, a plurality of the iPS cells are produced from a plurality of the somatic cells.

In some embodiments, the method further comprises isolating a population of the iPS cells (e.g., wherein at least 5%, 10%, 15%, 20%, 25%, 30%, 35%, 50%, 75% or greater of the subject cell type).

In some embodiments, the efficiency of converting somatic cells to iPS cells is at least 0.001%, 0.01%, 0.1%, 1% or greater.

In some embodiments, the method further comprises implanting the iPS cells in to a subject.

In some embodiments, the subject is suffering from a disorder.

In some embodiments, the iPS cells are from a donor different than the subject (e.g., a relative of the subject).

In another aspect, the invention features an iPS cell produced by a method comprising treating the somatic cell with at least two transcription factors and contacting the somatic cell with a DNA methyl transferase inhibitor or an HDAC inhibitor under conditions sufficient to produce an iPS cell from the somatic cell.

In some embodiments, the somatic cell is treated with at least two transcription factors prior to the step of contacting the somatic cell with the DNA methyl transferase inhibitor or the HDAC inhibitor.

In some embodiments, the step of treating the somatic cell with at least two transcription factors comprises treating the somatic cell with at least one heterologous nucleic acid sequence encoding at least two transcription factors.

In some embodiments, the somatic cell is treated with at least one heterologous nucleic acid sequence encoding at least two transcription factors by infection.

In some embodiments, the HDAC inhibitor comprises VPA.

In some embodiments, the transcription factors comprise Oct4, Sox2, Klf4 and c-Myc.

In some embodiments, the transcription factors comprise Oct4 and Sox2.

In some embodiments, the transcription factors comprise Oct4, Sox2 and Klf4.

In one aspect, the invention features a cell expressing Oct4, Sox2, Klf4 and c-Myc, comprising a DNA methyl transferase inhibitor, or an HDAC inhibitor, or a combination thereof.

In another aspect, the invention features a cell expressing Oct4, Sox2 and Klf4, comprising a DNA methyl transferase inhibitor, an HDAC inhibitor, or a combination thereof.

In yet another aspect, the invention features a cell expressing Oct4 and Sox2, comprising a DNA methyl transferase inhibitor, or an HDAC inhibitor, or a combination thereof.

In one aspect, the invention features a reaction mixture comprising a more primitive precursor or a less differentiated cell, e.g., a pluripotent stem cell (or a population thereof) compared to a somatic cell from which it was derived, and an exogenously produced DNA methyl transferase inhibitor or HDAC inhibitor, or a combination thereof.

In some embodiments, the less differentiated cell is an iPS cell.

In some embodiments, the iPS cell is produced by a method comprising treating the somatic cell with at least two transcription factors and contacting the somatic cell with a DNA methyl transferase inhibitor or an HDAC inhibitor under conditions sufficient to produce an iPS cell from the somatic cell.

In one aspect, the invention features a composition comprising an iPS cell produced by a method comprising treating the somatic cell with at least two transcription factors and contacting the somatic cell with a DNA methyl transferase inhibitor or an HDAC inhibitor under conditions sufficient to produce an iPS cell from the somatic cell.

In some embodiment, the somatic cell is treated with at least two transcription factors prior to the step of contacting the somatic cell with the DNA methyl transferase inhibitor or the HDAC inhibitor.

In some embodiments, the step of treating the somatic cell with at least two transcription factors comprises treating the somatic cell with at least one heterologous nucleic acid sequence encoding at least two transcription factors.

In some embodiments, the somatic cell is treated with at least one heterologous nucleic acid sequence encoding at least two transcription factors by infection.

In some embodiments, the HDAC inhibitor comprises VPA.

In some embodiments, the transcription factors comprise Oct4, Sox2, Klf4 and c-Myc.

In one aspect, the invention features a kit comprising: a somatic cell; at least one compound selected from a DNA methyl transferase inhibitor or an HDAC inhibitor, or a combination thereof; at least two transcription factors selected from the group consisting of Oct4, Sox2, Klf4 and c-Myc; and instructions for producing an iPS cell from a somatic cell.

In some embodiments, the HDAC inhibitor comprises VPA.

In some embodiments, the somatic cell is a human somatic cell.

In some embodiments, the somatic cell is selected from a fibroblast (e.g., primary fibroblast), a muscle cell (e.g., a myocyte), a cumulus cell, a neural cell, a liver cell, a GI tract cell, a mammary cell, a kidney cell, a blood cell, a vascular cell, a skin cell, an immune system cell, a lung cell, a bone cell, or a pancreatic islet cell.

In some embodiments, the somatic cell is a primary cell or is a progeny of a primary or secondary cell.

In some embodiments, the somatic cell is obtained from a sample selected from a group consisting of hair follicle, a blood sample, a swab sample and an adipose biopsy.

In some embodiments, the somatic cell is a healthy cell or a cell containing at least one genetic lesion.

In some embodiments, the kit further comprises a component for the detection of a marker for an iPS cell selected from a group selected from a group consisting of alkaline phophatase, NANOG, OCT4, SOX2, SSEA4, TRA-1-60 and TRA-1-81.

In some embodiments, the kit further comprises an iPS cell wherein the iPS cell is produced from the same cell type of the somatic cell.

In some embodiments, the kit further comprises a component for preparation of a karyotype from a cell.

In another aspect, the invention features a kit comprising an iPS cell produced by a method comprising treating the somatic cell with at least two transcription factors and contacting the somatic cell with a DNA methyl transferase inhibitor or an HDAC inhibitor under conditions sufficient to produce an iPS cell from the somatic cell.

In some embodiments, the HDAC inhibitor comprises VPA.

In some embodiments, the iPS cell is an isolated iPS cell.

In some embodiments, the iPS cell is frozen or in culture.

In yet another aspect, the invention features a kit comprising: an iPS cell produced by a method comprising treating the somatic cell with at least two transcription factors and contacting the somatic cell with a DNA methyl transferase inhibitor or an HDAC inhibitor under conditions sufficient to produce an iPS cell from the somatic cell; at least one component for directing the iPS cell to a differentiated cell; and instructions for directing the iPS cell to a differentiated cell.

In some embodiments, the HDAC inhibitor comprises VPA.

In some embodiments, the iPS cell is an isolated iPS cell.

In some embodiments, the iPS cell is frozen or in culture.

In some embodiments, the differentiated cell comprises a fibroblast (e.g., primary fibroblast), a muscle cell (e.g., a myocyte), a cumulus cell, a neural cell, a liver cell, a GI tract cell, a mammary cell, a kidney cell, a blood cell, a vascular cell, a skin cell, an immune system cell, a lung cell, a bone cell, or a pancreatic islet cell.

In one aspect, the invention features a kit comprising: an iPS cell produced by a method comprising treating the somatic cell with at least two transcription factors and contacting the somatic cell with a DNA methyl transferase inhibitor or an HDAC inhibitor under conditions sufficient to produce an iPS cell from the somatic cell; at least one component for expanding the iPS cell; and instructions for expanding the iPS cell.

In some embodiments, the HDAC inhibitor comprises VPA.

In some embodiments, the iPS cell is an isolated iPS cell.

In some embodiments, the iPS cell is frozen or in culture.

In one aspect, the invention features a method of instructing an end-user to produce an iPS cell from a somatic cell, the method comprises providing at least one of the components or a kit described herein; and instructing the end-user using an information material, e.g., a printed material or a computer readable material, or both.

In another aspect, the invention features a method of instructing an end-user to produce a differentiated cell from an iPS cell, the method comprises providing at least one of the components or a kit described herein; and instructing the end-user using an information material, e.g., a printed material or a computer readable material, or both.

In yet another aspect, the invention features a method of instructing an end-user to expand an iPS cell, the method comprises providing at least one of the components or a kit described herein; and instructing the end-user using an information material, e.g., a printed material or a computer readable material, or both.

In one aspect, the invention features a reaction mixture comprising a cell expressing Oct4, Sox2, Klf4 and c-Myc; and a DNA methyl transferase inhibitor, or an HDAC inhibitor, or a combination thereof.

In another aspect, the invention features a reaction mixture comprising a cell expressing Oct4, Sox2 and Klf4; and a DNA methyl transferase inhibitor, or an HDAC inhibitor, or a combination thereof.

In yet another aspect, the invention features a reaction mixture comprising a cell expressing Oct4 and Sox2; and a DNA methyl transferase inhibitor, or an HDAC inhibitor, or a combination thereof.

In one aspect, the invention features a composition comprising a cell expressing Oct4, Sox2, Klf4 and c-Myc; and a DNA methyl transferase inhibitor, or an HDAC inhibitor, or a combination thereof.

In another aspect, the invention features a composition comprising a cell expressing Oct4, Sox2 and Klf4; and a DNA methyl transferase inhibitor, or an HDAC inhibitor, or a combination thereof.

In yet another aspect, the invention features a composition comprising a cell expressing Oct4 and Sox2; and a DNA methyl transferase inhibitor, or an HDAC inhibitor, or a combination thereof.

Accordingly, in one aspect, the disclosure features a method of producing a more primitive precursor or a less differentiated cell, e.g., pluripotent stem cell (or a population thereof) from a somatic cell, or reprogramming a somatic cell. The method comprises:

contacting a somatic cell with a DNA methyl transferase inhibitor or a histone deactylase (HDAC) inhibitor (e.g., VPA), or a combination thereof, to thereby produce a primitive precursor or a less differentiated cell, e.g., pluripotent stem cell (or a population thereof) or to reprogram the somatic cell. In some embodiments, the HDAC inhibitor selectively inhibits a Class I or Class II HDAC. In some preferred embodiments, the method includes contacting a somatic cell with VPA.

In one embodiment, the somatic cell further expresses, or has increased expression, of one or more transcription factor(s) (e.g., two, three, or four transcription factors). In one embodiment, the transcription factor is one or more of Oct4, Klf4, Sox2 and c-Myc. In one embodiment, the somatic cell does not express c-Myc or does not express c-Myc at statistically significant levels or does not over express c-Myc. In one embodiment, the somatic cell does not express c-Myc or Klf4 or does not express c-Myc or Klf4 at statistically significant levels or does not over express c-Myc and Klf4. In some embodiments, the somatic cell can express, e.g., Oct4 and Sox2 or the somatic cell can express, e.g., Oct4, Klf4 and Sox2 or the somatic cell can express Oct4, Klf4, Sox2 and c-Myc.

In one embodiment, the somatic cell includes a heterologous nucleic acid sequence, e.g., a heterologous nucleic acid sequence encoding a transcription factor, e.g., a nucleic acid encoding a transcription factor described herein. In one embodiment, the nucleic acid encodes Oct4, Klf4, Sox2 or c-Myc. In one embodiment, the somatic cell includes two or more heterologous nucleic acid sequences, e.g., encoding transcription factors, e.g., encoding two or more of Oct4, Klf4, Sox2 and c-Myc. In one embodiment, the somatic cell includes at least three heterologous nucleic acid sequences, e.g., encoding Oct4, Klf4 and Sox2. In one embodiment, somatic cell includes at least two heterologous nucleic acid sequences, e.g., encoding Oct4 and Sox2. In another embodiment, the somatic cell includes at least four heterologous nucleic acid sequences, e.g., encoding Oct4, Klf4, Sox2 and c-Myc. In one embodiment, the nucleic acid sequence is introduced into the somatic cell, or the somatic cell is the progeny of such a somatic cell. In an embodiment the cell does not include a heterlogous c-Myc gene. In an embodiment the cell does not include heterologous c-Myc and Klf4 genes. In an embodiment the somatic cell is human and any heterologous gene, e.g., transcription factor gene, is human as well, e.g., the human equivalent of any of Oct4, Klf4, Sox2 and c-Myc. In an embodiment the method includes the further step of selecting a more primitive precursor or a less differentiated cell, e.g., pluripotent stem cell (or a population thereof) made by the method which has lost a vector which encodes the heterologous nucleic acid.

In one embodiment, the somatic cell is contacted with a DNA methyltransferase inhibitor, e.g., a DNA methyltransferase inhibitor described herein. In one embodiment, the DNA methyltransferase inhibitor is 5 azacytidine.

In one embodiment, the somatic cell is contacted with a HDAC inhibitor, e.g., a HDAC inhibitor described herein. In one embodiment, the HDAC inhibitor is one or more of valproic acid (VPA), suberoylanilide hydroxamic acid (SAHA) and trichstatin A (TSA). In a preferred embodiment, the method includes contacting a somatic cell with VPA.

In one embodiment, dexamethasone is administered in combination with the DNA methyl transferase inhibitor (e.g., 5-azacytidine) or the histone deactylase (HDAC) inhibitor, or the combination thereof.

In one embodiment, the somatic cell is selected from a fibroblast (e.g., primary fibroblast), a muscle cell (e.g., a myocyte), a cumulus cell, a neural cell, a liver cell, a GI tract cell, a mammary cell, a hepatocyte and a pancreatic islet cell. In one embodiment, the somatic cell is a primary cell line or is the progeny of a primary or secondary cell line. In one embodiment, the somatic cell is obtained from a sample, e.g., a hair follicle, a blood sample, a swab sample or an adipose biopsy.

In an embodiment, the somatic cell is obtained from a first individual and the more primitive precursor or a less differentiated cell, e.g., pluripotent stem cell (or a population thereof) (or a tissue derived therefrom) is administered to the same first individual, or to a second individual, e.g., an individual related to said first individual. The second individual can be an individual who carries a different allele for a selected gene than does the first individual. E.g., the first individual can have an allele which does not cause a disease state or unwanted condition and the second individual has the allele which causes the disease state or unwanted condition.

In one embodiment, the number of stem cells produced, e.g., in the presence of a DNA methyl transferase inhibitor or a histone deactylase (HDAC) inhibitor, or a combination thereof, is 5-, 10-, 15-, 20-, 25-, 30-, 35-, 40-, 50-, 100-, 120-, 130-, 140-, 150-, 200-, 250-, 500-, 750- or 1000- fold greater than the number of stem cells produced by alternative methods, e.g., the number of stem cells produced by cell expressing one or more transcription factors, e.g., Oct4 and Sox2, or Oct4, Klf4 and Sox2 or Oct4, Klf4, Sox2 and c-Myc, or the number of stem cells produced in the absence of a DNA methyl transferase inhibitor or a histone deactylase (HDAC) inhibitor, or a combination thereof.

In another aspect, the disclosure features a population of cells, e.g., pluripotent stem cell or a population of pluripotent stem cells, produced by a method described herein.

In another aspect, the invention features, a reaction mixture including a somatic cell and a sufficient amount of DNA methyl transferase inhibitor or a histone deactylase (HDAC) inhibitor such as VPA, or a combination thereof, to convert the somatic cell to a more primitive precursor or a less differentiated cell, e.g., pluripotent stem cell (or a population thereof). In one embodiment, the somatic cell is treated with one or more transcription factors, for example, a transcription factor selected from Oct4, Klf4, Sox2 and c-Myc. In some embodiments, the somatic cell is treated with 2, 3 or 4 transcription factors (e.g., the somatic cell is treated with Oct4 and Sox2, the somatic cell is treated with Oct4, Sox2, and Klf4 or the somatic cell is treated with Oct4, Sox2, Klf4, and c-Myc). In some embodiments, the somatic cell is not treated with c-Myc and/or Klf4.

In another aspect, the disclosure features a composition, e.g., a pharmaceutical composition, comprising a cell, e.g., a pluripotent stem cell or a population of pluripotent stem cells, produced by a method described herein.

The methods and pluripotent stem cells described herein are useful for treating a wide variety of conditions, including hematopoietic conditions (e.g., sickle cell anemia, leukemias, immune deficiencies), cardiac disorders (e.g., myocardial infarcts, and myopathies) and disorders such as liver disease, diabetes, thyroid abnormalities, neurodegenerative/neurological disorders (e.g., Parkinson's, Alzheimer's, stroke injuries, spinal chord injuries), circulatory disorders, respiratory disorders, wound healing and/or repair, bone repair, and enzyme abnormalities.

In one embodiment, the disclosure features a method of treating a disorder described herein, wherein the method includes: administering a pluripotent stem cell or a population of pluripotent stem cells produced by a method described herein to a subject, e.g., a subject that suffers from a disorder described herein.

In one embodiment of the methods described herein, the somatic cell contains one or more genetic defect, and, e.g., the pluripotent stem cell produced by a method described herein includes the genetic defect or defects. In some embodiments, the genetic defect is corrected (e.g., by homologous recombination) in the pluripotent stem cell, e.g., to provide a corrected pluripotent stem cell. Such cells can be administered by known methods such as the methods described e.g., in U.S. Publication No: 20030228293, the contents of which is incorporated herein by reference. The genetic defect corrected can be, for example, a genetic defect that causes an immune system disorder; a genetic defect that causes a neurological disorder; a genetic defect that causes a cardiac disorder; a genetic defect that causes a circulatory disorder; a genetic defect that causes a metabolic disorder such as diabetes; or a genetic defect that causes a respiratory disorder.

In some embodiments of the methods described herein, the pluripotent stem cell or population of pluripotent stem cells are differentiated in vitro into tissue or cell types useful in treating the condition or disorder. In one embodiment, the pluripotent stem cell or tissues or cell types derived from the pluripotent stem cells are introduced into the subject from which the somatic cell was obtained. In one embodiment, the somatic cell is obtained from a subject having one or more genetic defects and the corrected pluripotent stem cell or a tissue of cell type derived from the corrected pluripotent stem cell is reintroduced to the subject. Differentiation can be effected by known methods. In one embodiment, the pluripotent stem cells are used to produce hematopoietic stem cells (HSC) which are, e.g., useful for transplantation and restoration of immune function in immune deficient recipients.

The methods described herein can further include maintaining the pluripotent stem cells under conditions which result in their differentiation into a desired cell type(s) (e.g., into repaired neurons, cardiac myocytes, blood cell type, bone cell (e.g., osteoblast) or pancreatic cells).

In one aspect, the invention includes a stem cell (e.g., an iPS) described herein for the manufacture of a medicament for treating a disorder described herein. The medicament can include other features described herein.

Kits for practicing methods disclosed herein and for making cells disclosed herein (e.g., iPS cells) are included.

In one aspect, a kit will contain a somatic cell, a component described herein (e.g., VPA) and instructions for converting a somatic cell to an iPS cell using the method described herein.

In one embodiment, the somatic cell is directed to an iPS cell. In one embodiment, the somatic cell can be used as a control.

In one embodiment, a kit will contain at least one of the components listed below. In one preferred embodiment, the kit contains at least two of the components listed below. Any combination of the components described herein can be provided. For example, any combination of 2, 3, 4, 5 or 6 of the components described herein can be provided.

Exemplary components include the compounds described herein, e.g., a composition(s) that includes a compound(s) described herein, e.g., at least one compound selected from a DNA methyl transferase inhibitor or an HDAC inhibitor (e.g., VPA), e.g., a DNA methyl transferase inhibitor or an HDAC inhibitor described herein. The compound can be provided in a watertight or gas tight container which in some embodiments is substantially free of other components of the kit. The compound can be supplied in more than one container, e.g., it can be supplied in a container having sufficient reagent for a predetermined number of conversions, e.g., 1, 2, 3 or greater. A compound(s) described herein (e.g., VPA) can be provided in any form, e.g., liquid, dried or lyophilized form. It is preferred that a compound(s) described herein be substantially pure and/or sterile. When a compound(s) described herein is provided in a liquid solution, the liquid solution preferably is an aqueous solution, with a sterile aqueous solution being preferred. When a compound(s) described herein is provided as a dried form, reconstitution generally is by the addition of a suitable solvent. The solvent, e.g., sterile water or buffer, can optionally be provided in the kit.

The kit can include a transcription factor, e.g., a transcription factor or combination of transcription factors described herein, e.g., one or more of Oct4, Klf4, Sox2 or c-Myc or a nucleic acid encoding the same transcription factor. For example, the kit can provide a vector, e.g., a plasmid or a viral vector, e.g., a retroviral, a lentiviral or an adenoviral vector, which can express one or more of Oct4, Klf4, Sox2 or c-Myc. In some embodiments, the transcription factor is fused to a tag, e.g., a GFP tag, a YFP tag or a RFP tag.

The kit can include a component for the detection of a marker for iPS cells, e.g., for a marker described herein, e.g., a reagent for the detection of alkaline phosphatase (AP), NANOG, OCT4, SOX2, SSEA4, TRA-1-60 or TRA-1-81, e.g., an antibody against the marker or primers for a RT-PCR or PCR reaction, e.g., a semi-quantitative or quantitative RT-PCR or PCR reaction. Such markers can be used to evaluate whether an iPS cell has been produced. If the detection reagent is an antibody, it can be supplied in dry preparation, e.g., lyophilized, or in a solution. The antibody or other detection reagent can be linked to a label, e.g., a radiological, fluorescent or colorimetric label for use in detection. If the detection reagent is a primer, it can be supplied in dry preparation, e.g., lyophilized, or in a solution.

It may be desirable to perform an analysis of the karyotype of the iPS cell. Accordingly, the kit can include a component for karyotyping, e.g., a probe, a dye, a substrate, an enzyme, an antibody or other useful reagents for preparing a karyotype from a cell.

The kit can also include an iPS cell, e.g., an iPS cell derived from the same cell type as the somatic cell. In one embodiment, the iPS cell can be for use as a control.

The kit can also include informational materials, e.g., instructions, for use of two or more of the components included in the kit.

The informational material can be descriptive, instructional, marketing or other material that relates to the methods described herein and/or the use of a compound(s) described herein for the methods described herein. In one embodiment, the informational material can include information about production of the compound, molecular weight of the compound, concentration, date of expiration, batch or production site information, and so forth. In one embodiment, the informational material relates to methods for culturing the compound. In one embodiment, the informational material can include instructions to culture a compound(s) (e.g., a HDAC inhibitor(s) such as VPA and/or a DNA methyltransferase inhibitor(s)) described herein in a suitable manner to perform the methods described herein, e.g., in a suitable dose, dosage form, or mode of administration (e.g., a dose, dosage form, or mode of administration described herein) (e.g., to a cell in vitro or a cell in vivo). In another embodiment, the informational material can include instructions to administer a compound(s) described herein to a suitable subject, e.g., a human, e.g., a human having or at risk for a disorder described herein or to a cell in vitro.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

Some specific embodiments will provide a somatic cell; at least one compound selected from a DNA methyl transferase inhibitor or an HDAC inhibitor (e.g., VPA), e.g., a DNA methyl transferase inhibitor or an HDAC inhibitor described herein; a transcription factor, e.g., a transcription factor or combination of transcription factors described herein, e.g., one or more of Oct4, Klf4, Sox2 or c-Myc or a nucleic acid encoding the same transcription factor; and instructions for use of one or more of the components included in the kit.

In some embodiments, the kit further includes a component for the detection of a marker for iPS cells, e.g., for a marker described herein, e.g., a reagent for the detection of alkaline phophatase, NANOG, OCT4, SOX2, SSEA4, TRA-1-60 or TRA-1-81, e.g., an antibody against the marker.

In some embodiments, the kit further includes an iPS cell, e.g., an iPS cell derived from the same cell type as the somatic cell.

In another embodiment, the kit further includes a component for preparation of a karyotype from a cell.

In one embodiment, the somatic cell is a human somatic cell.

In one embodiment, the somatic cell is selected from a fibroblast (e.g., primary fibroblast), a muscle cell (e.g., a myocyte), a cumulus cell, a neural cell, a liver cell (e.g., a hepatocyte), a GI tract cell, a mammary cell, a kidney cell, a blood cell, a vascular cell, a skin cell, an immune system cell (e.g., a lymphocyte), a lung cell, or a pancreatic islet cell.

In one embodiment, the somatic cell is a primary cell line or is the progeny of a primary or secondary cell line.

In one embodiment, the somatic cell is obtained from a sample, e.g., a hair follicle, a blood sample, a swab sample or an adipose biopsy.

In one embodiment, the somatic cell is a healthy cell or a cell containing one or more genetic lesion(s).

In another aspect, a kit contains an iPS cell made by a method described herein, e.g., using one or more component(s) described herein (e.g., VPA).

In one embodiment, the iPS cell is an isolated iPS cell.

In one embodiment, the iPS cell is frozen or in culture.

In another aspect, the invention features a kit comprising an iPS cell made by a method described herein and one or more component(s) for expanding (e.g., multiplying or proliferating) the iPS cell. In some embodiments, the kit comprises one or more component(s) for culturing an iPS cell in media thereby expanding the iPS cell. In one embodiment, the kit comprises a feeder layer, e.g., an irradiated MEF feeder layer. In one embodiment, the kit comprises hES cell media e.g., hES cell media containing Knockout DMEM supplemented with 10% knockout serum replacement, 10% human plasma fraction, 10 ng/ml bFGF, nonessential amino acids, β-mercaptoethanol, L-glutamine, and/or penicillin/streptomycin. In one embodiment, hES cell media further contain a chemical ROCK (p160-Rho-associated coiled-coil kinase) inhibitor e.g., Y-27632 (see e.g., Watanabe, K. et al. A ROCK inhibitor permits survival of dissociated human embryonic stem cells. Nat. Biotechnol.; 25, 681-686 (2007). In some embodiments, the ROCK inhibitor is at a concentration of from about 1 uM to about 100 um (e.g., at a concentration of e.g., 10 uM). In some embodiments, the ROCK inhibitor is provided in the media for at least about 1 day e.g., for the first two days after passage. In some embodiments, the ROCK inhibitor increases the seeding efficiency of the iPS cell.

In another aspect, a kit contains an iPS cell, for example, made by a method described herein and instructions for directing an iPS cell to a differentiated cell.

In one embodiment, the iPS cell is made by using one or more component(s) described herein (e.g., VPA).

In one embodiment, the differentiated cell is directed from an iPS cell by a method, for example, described in the art. Exemplary methods described in the art include, Dimos J T, et al., Induced Pluripotent Stem Cells Generated from Patients with ALS Can Be Differentiated into Motor Neurons. Science. 2008; 321(5893):1218-21; Mauritz C, et al., Generation of functional murine cardiac myocytes from induced pluripotent stem cells. Circulation. 2008; 118(5):507-17; Sharma A D, et al., Murine embryonic stem cell-derived hepatic progenitor cells engraft in recipient livers with limited capacity of liver tissue formation. Cell Transplant. 2008; 17(3):313-23; Toh W S, et al., Differentiation of human embryonic stem cells toward the chondrogenic lineage. Methods Mol Biol. 2007; 407:333-49; Vodyanik M A, et al., Directed differentiation of human embryonic stem cells to dendritic cells. Methods Mol Biol. 2007; 407:275-93; Roche E, et al., Insulin producing cells from embryonic stem cells: experimental considerations. Methods Mol Biol. 2007; 407:295-309. Each of these references are incorporated herein by reference.

In another embodiment, the differentiated cell is selected from a fibroblast (e.g., primary fibroblast), a muscle cell (e.g., a myocyte), a cumulus cell, a neural cell, a liver cell (e.g., a hepatocyte), a GI tract cell, a mammary cell, a kidney cell, a blood cell, a vascular cell, a skin cell, an immune system cell (e.g., a lymphocyte), a lung cell, a bone cell, or a pancreatic islet cell.

The kit can provide buffers e.g., reaction buffers, solvents, diluents, solutions, stabilizers, preservatives, media, cell lines, vectors, enzymes, secondary antibodies and other materials useful for practicing the methods e.g., a packaging cell line or a packaging vector for virus production, media for culturing iPS cells, or a secondary antibody used for Western analysis or immunofluorescence staining. Alternatively, the other ingredients can be included in the kit, but in different compositions or containers than a compound described herein. In such embodiments, the kit can include instructions for admixing a compound(s) described herein and the other ingredients, or for using a compound(s) described herein together with the other ingredients, e.g., instructions on combining the two agents prior to administration.

The kit will typically be provided with its various elements included in one package, e.g., a fiber-based, e.g., a cardboard, or polymeric, e.g., a styrofoam box. The enclosure can be configured so as to maintain a temperature differential between the interior and the exterior, e.g., it can provide insulating properties to keep the reagents at a preselected temperature for a preselected time.

The kit can include one or more containers for the composition containing a compound(s) described herein. In some embodiments, the kit contains separate containers (e.g., two separate containers for the two agents), dividers or compartments for the composition(s) and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

In one aspect, the invention features a method for reprogramming a somatic cell to form a less differentiated cell comprising contacting the somatic cell with DNA methyl transferase inhibitor or an HDAC inhibitor under conditions sufficient to reprogram the somatic cell thereby producing a cell that is less differentiated than the somatic cell (e.g., an ES-like cell).

In some embodiments, the DNA methyl transferase inhibitor comprises 5′-azacytidine.

In some embodiments, the HDAC inhibitor selectively inhibits a Class I or Class II HDAC.

In some embodiments, the HDAC inhibitor comprises VPA, SAHA or TSA, or a combination thereof.

In some embodiments, the HDAC inhibitor comprises VPA.

In another aspect, the invention features a cell produced by a method comprising contacting the somatic cell with DNA methyl transferase inhibitor or an HDAC inhibitor under conditions sufficient to reprogram the somatic cell.

In yet another aspect, the invention features a reprogrammed somatic cell in which expression of a plurality of genes that are up-regulated or down-regulated in ES cells is up- or down-regulated in the reprogrammed somatic cell, wherein these genes are not up- or down-regulated in the somatic cell prior to reprogramming

In some embodiments, expression of the genes Rex3 and Zfp7 is up-regulated, and expression of the genes Aspn and Meox2 are down-regulated compared to the expression of these genes in the somatic cell prior to reprogramming

In one aspect, the invention features a reprogrammed somatic cell in which expression of a plurality of genes that are specifically expressed in ES cells are up-regulated in the reprogrammed somatic cell, wherein these genes are not up-regulated in the somatic cell prior to reprogramming

In another aspect, the invention features a reprogrammed somatic cell in which expression of a plurality of genes that are specifically expressed in the somatic cells prior to reprogramming, but are not expressed in ES cells, are down-regulated in the reprogrammed somatic cell, wherein these genes are not down-regulated in the somatic cell prior to reprogramming

In yet another aspect, the invention features a reaction mixture comprising a somatic cell and (i) a DNA methyl transferase inhibitor, (ii) an HDAC inhibitor, or (iii) a mixture thereof.

In some embodiments, the HDAC inhibitor is VPA.

As used herein, the term histone deacetylase (HDAC) refers to a histone deacetylase Class I and/or Class II enzyme. Exemplary HDACs are disclosed, for example, in US 20070093413, which is incorporated herein by reference.

As used herein, the term HDAC inhibitor refers to a compound that inhibits a histone deacetylase Class I and/or Class II enzyme. In some embodiments, the compound selectively inhibits a Class I or Class II HDAC.

By “selective” is meant at least 20%, 50%, 75%, 2-fold, 3-fold, 4-fold, 5-fold, 6-fold, or 10-fold greater inhibition of an HDAC over another enzyme, for example a Class III or Class IV histone deacetylase. Thus, in some embodiments, the agent is selective for HDAC over a Class III histone deacetylase. In some embodiment the inhibitor is specific for a Class I or Class II and thus does not significantly inhibit HDACs of other classes.

As used herein, a heterologous nucleic acid, is a nucleic acid other than a native endogenous sequence for that gene. E.g., an additional copy of a gene inserted into a chromosome, or a copy on a vector, e.g., a replicative on non replicative vector which has not integrated into the chromosome.

Other features and advantages of the instant invention will become more apparent from the following detailed description and claims. Embodiments of the invention can include any combination of features described herein. In no case does the term “embodiment” necessarily exclude one or more other features disclosed herein, e.g., in another embodiment. The contents of all references, patent applications and patents, cited throughout this application are hereby expressly incorporated by reference.

### BRIEF DESCRIPTION OF THE DRAWINGS

FIG. 1. Small molecules that improve reprogramming efficiency in 4-factor infected MEFs.

FIG. 1(a) MEFs infected with the four factors (Oct4, Sox2, Klf4 and c-Myc) were treated with various chemicals for a week, the percentage of Oct4-GFP positive cells induced was measured through FACS analysis, and compared to infected MEFs without treatment or treated with DMSO (the solvent for Dexamethasone, SAHA and TSA). The chemicals used were: 5′-azaC (2 μM), dexamethasone (dex, 1 μM), 5′-azaC (2 μM) together with dexamethasone (1 μM), VPA (2 mM), SAHA (5 μM) and TSA (20 nM). n=4 for each treatment. For all figures in this study, standard deviations are indicated by error bars, and P values by two-tailed student t-test less than 0.05, 0.01 and 0.001 are indicated by one, two and three asterisks respectively.

FIG. 1(b) Representative FACS plots from infected MEFs treated with 2 mM VPA compared to the control infected MEFs without VPA treatment.

FIG. 1(c) Representative pictures at 11 days post-infection showing a significant increase of GFP positive iPS colonies in infected MEFs with VPA treatment compared to non-treated control.

FIG. 2. Efficient induction of c-Myc-free iPS cells with chemical treatment.

FIG. 2(a) MEFs infected with Oct4, Sox2, Klf4 (but no c-Myc) were treated with 5′-azaC (2 μM) or VPA (2 mM) for a week, the percentage of Oct4-GFP positive cells induced was measured through FACS analysis at 10 days post-infection, and compared to infected MEFs without treatment. n=4 for each condition.

FIG. 2(b) Representative FACS plots from 5′-azaC and VPA treated MEFs infected with the three factors compared to the control infected MEFs without chemical treatment.

FIG. 2(c) Representative pictures at 16 days post-infection showing a significant increase of GFP positive iPS colonies in 3-factor infected MEFs with VPA treatment compared to non-treated control.

FIG. 3. iPS-m cells resembles ES cells in gene expression and pluripotency.

FIG. 3(a) mouse iPS-m cells exhibited typical ES cell morphology and expressed Oct4-GFP homogeneously.

FIG. 3(b) mouse iPS-m colonies exhibited high alkaline phosphatase activities.

FIG. 3(c) a cluster analysis dendrogram showing hierarchical clustering of mouse iPS-m lines (iPS-m1 was induced without VPA treatment; iPS-m18 and iPS-m23 were induced with VPA treatment), a mouse ES cell line (AV3) and MEFs based on transcriptional similarity.

FIG. 3(d) iPS-m1 (induced without VPA treatment) and iPS-m28 (induced with VPA treatment) both formed teratomas when injected into SCID mice. Hematoxylin and eosin staining of teratoma sections showed differentiation of iPS-m cells to various tissues, including skin, muscle, gland, cartilage, intestinal gland and neural rosettes.

FIG. 3(e) six iPS-m lines (iPS-m64 and iPS-m73 were induced without VPA treatment; iPS-m81, 82, 83 and 84 were induced with VPA treatment) were injected into blastocysts. All gave rise to high contribution chimeras. Shown here is lacZ staining of e10.5 chimeric embryo from donor iPS-m82 cells, in contrast to the absence of staining in the non-injected control. Similar results were obtained from all other iPS-m lines injected.

FIG. 3(f) Sections of chimeric embryos showed extensive contribution of injected cells to tissues derived from all three germ layers, including the neural tube (nt, ectoderm derivative), gut endoderm (g) and limb bud (lb, mesoderm derivative).

FIG. 4. VPA treatment alone partially reprograms MEFs.

(a) Genes that were specifically expressed in ES cells and MEFs (>10 fold difference) were selected, and scatter plot was generated to visualize the effect of VPA treatment on the expression of these genes. Each blue dots represent one gene, and the position of the blue dot indicate the relative expression level, the average signal (AVE_signal), in MEFs treated with VPA versus untreated. The thick red line indicates identical expression levels. The two adjacent red lines indicate positions where VPA treatment causes a two fold up or down-regulation of the gene expression.

(b) Examples of the relative levels of expression of ES cell-specific transcripts (Rex3 and Zfp7) in ES cells, iPS-m cells, untreated MEFs and MEFs treated with VPA.

(c) Examples of the relative levels of expression of MEF-specific transcripts (Aspn and Meox2) in ES cells, iPS-m cells, untreated MEFs and MEFs treated with VPA.

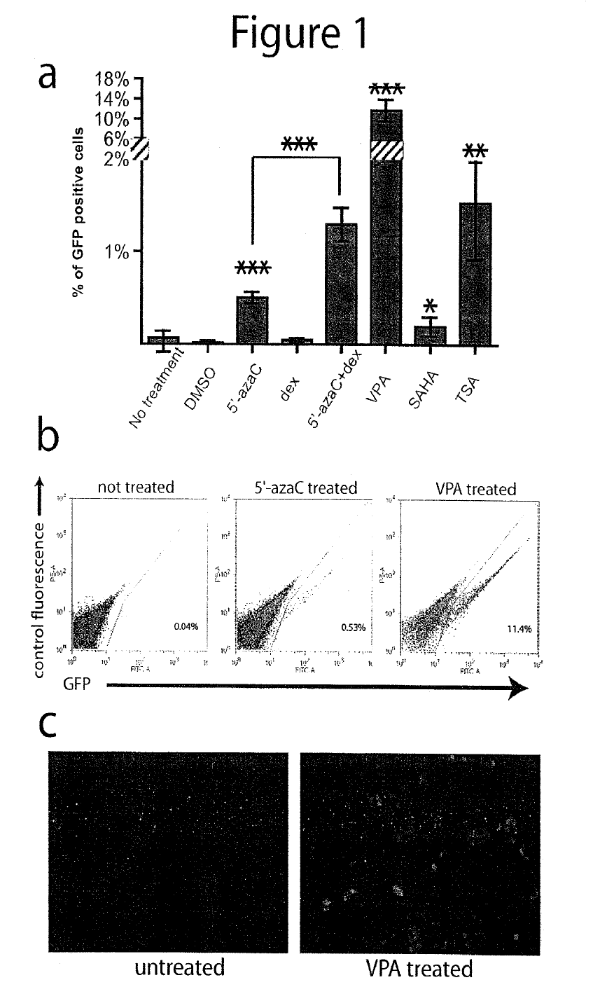

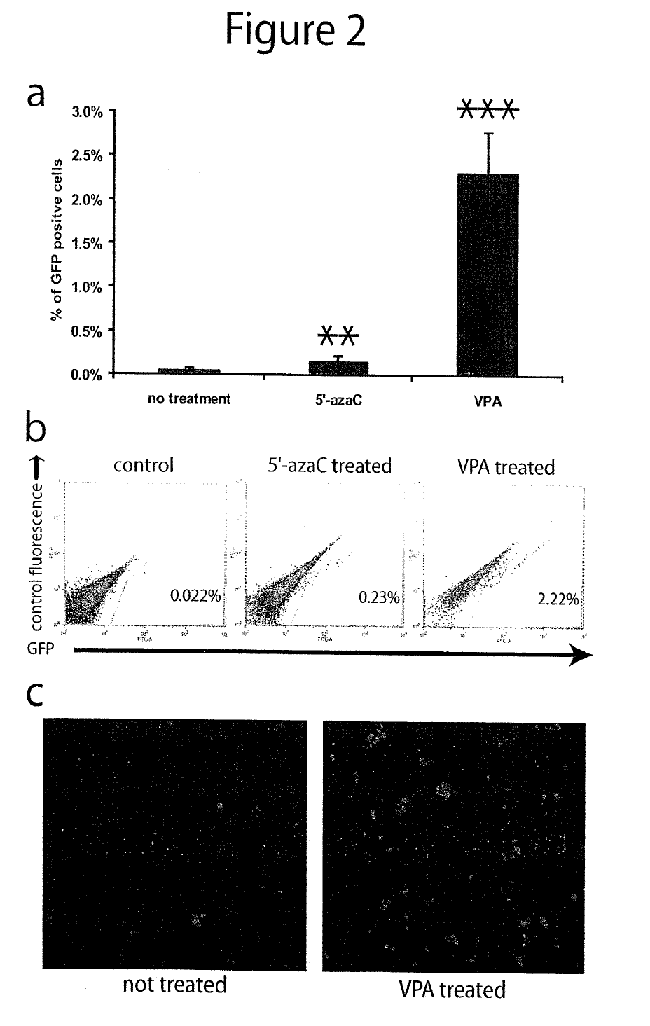

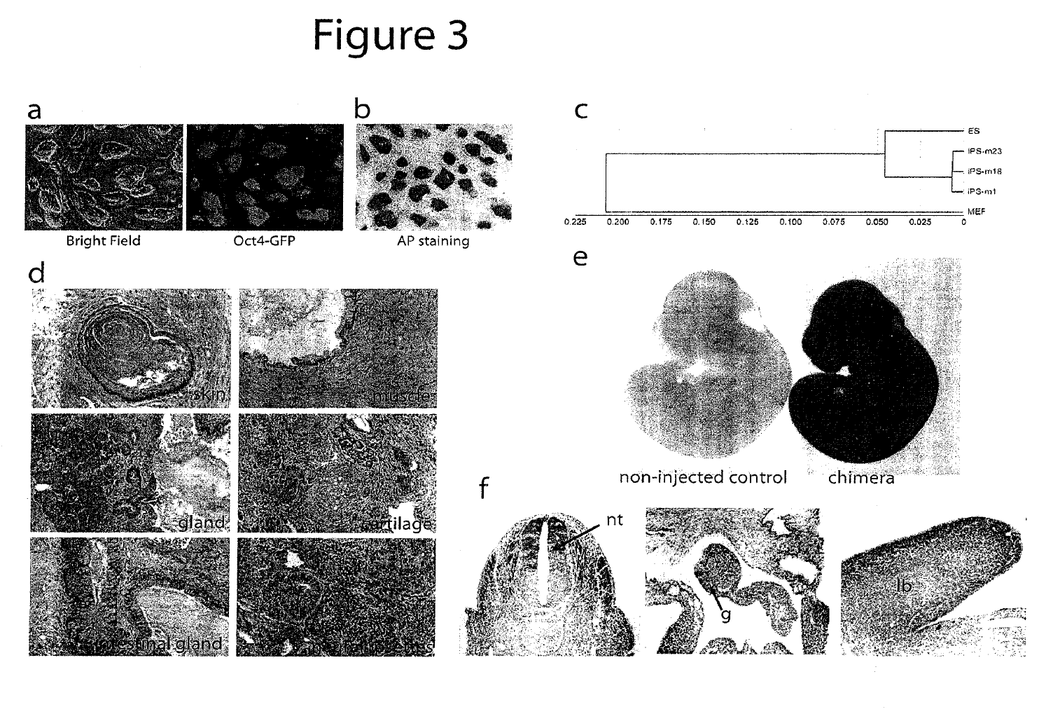

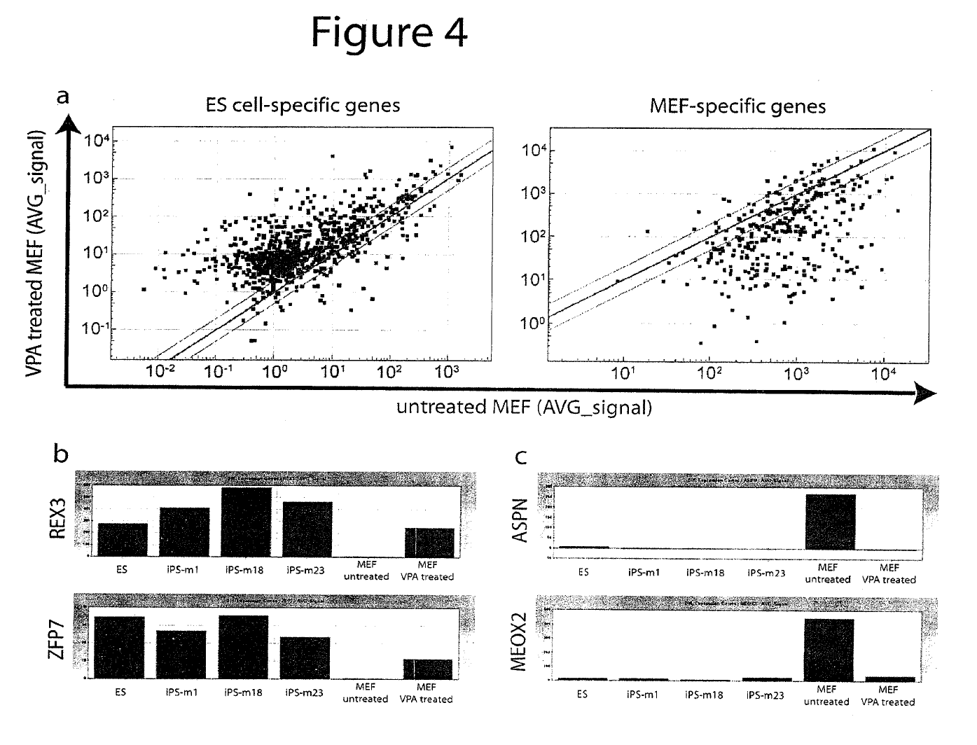

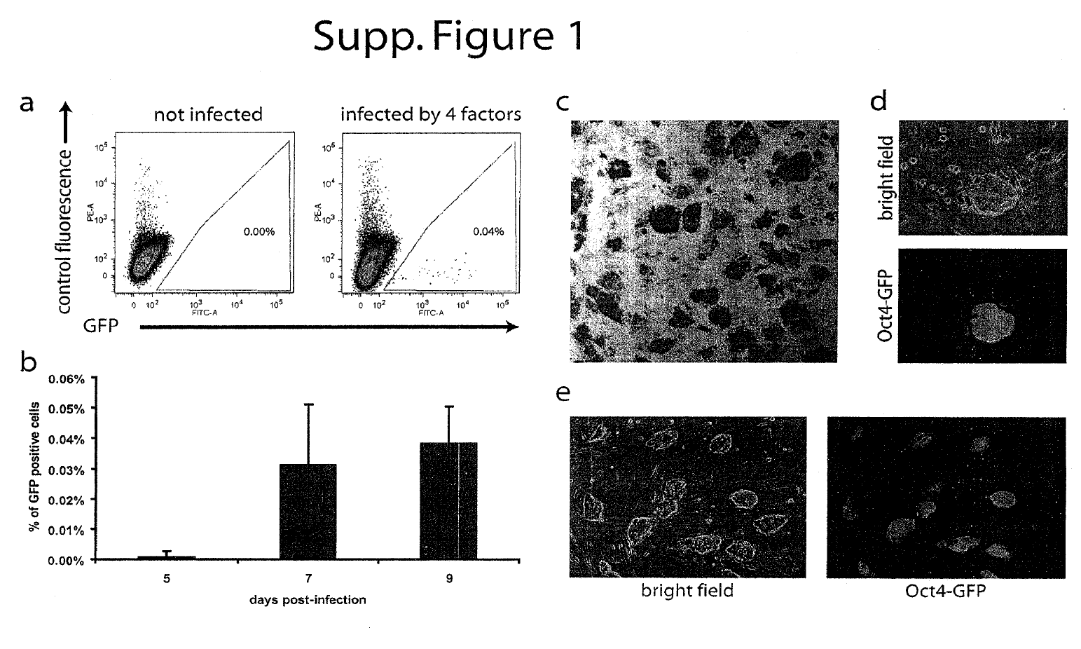

Supplementary FIG. 1. Reprogramming of Oct4-GFP/+MEF using four factors

Supplementary FIG. 1(a) and Supplementary FIG. 1(b) FACS analysis showed that around 0.04% Oct4-GFP positive cells were induced at 7 days post-infection, and the percentage of Oct4-GFP positive cells remained at around the same level at 9 days post-infection; while no GFP positive cells were induced in the non-infected control. n=4 for each time point. Error bars indicate standard deviation.

Supplementary FIG. 1(c) retroviral expression of Oct4, Sox2, Klf4 and c-Myc in MEFs induced ES-like colonies that could be identified by alkaline phosphatase staining.

Supplementary FIG. 1(d) iPS colonies had ES cell-like morphology and expressed Oct4-GFP.

Supplementary FIG. 1(e) iPS colonies were picked based on morphology and GFP expression, and iPS cell lines were established, which exhibited typical ES cell morphology and expressed Oct4-GFP homogeneously.

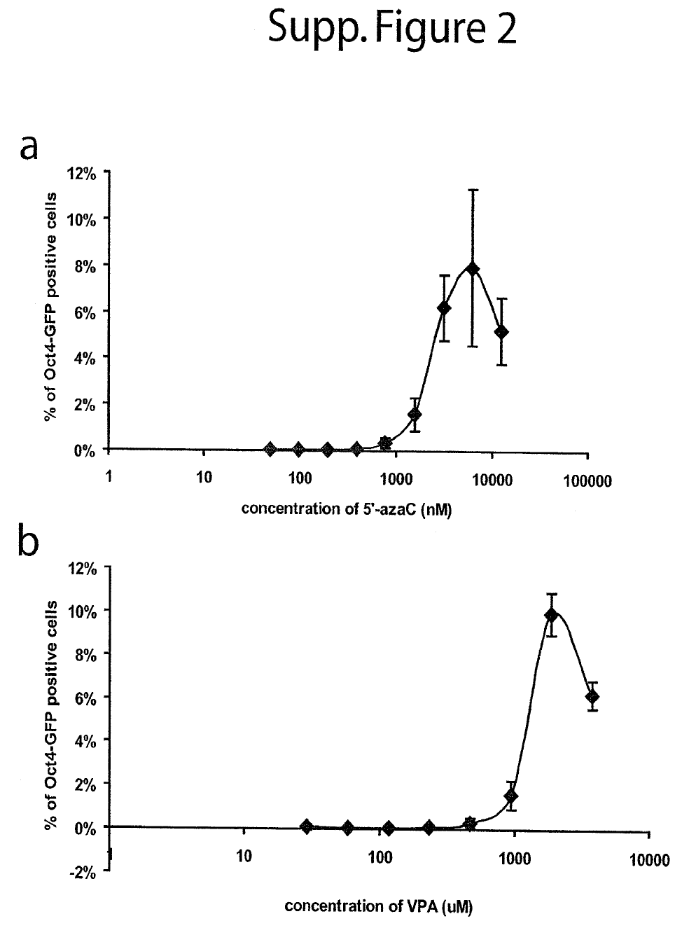

Supplementary FIG. 2. Dose response curves of the effect of 5′-azaC (Supplementary FIG. 2(a)) and VPA (Supplementary FIG. 2(b)) treatment on reprogramming efficiency measured by the percentage of Oct4-GFP positive cells induced. n=4 for each concentration. Error bars indicate standard deviation.

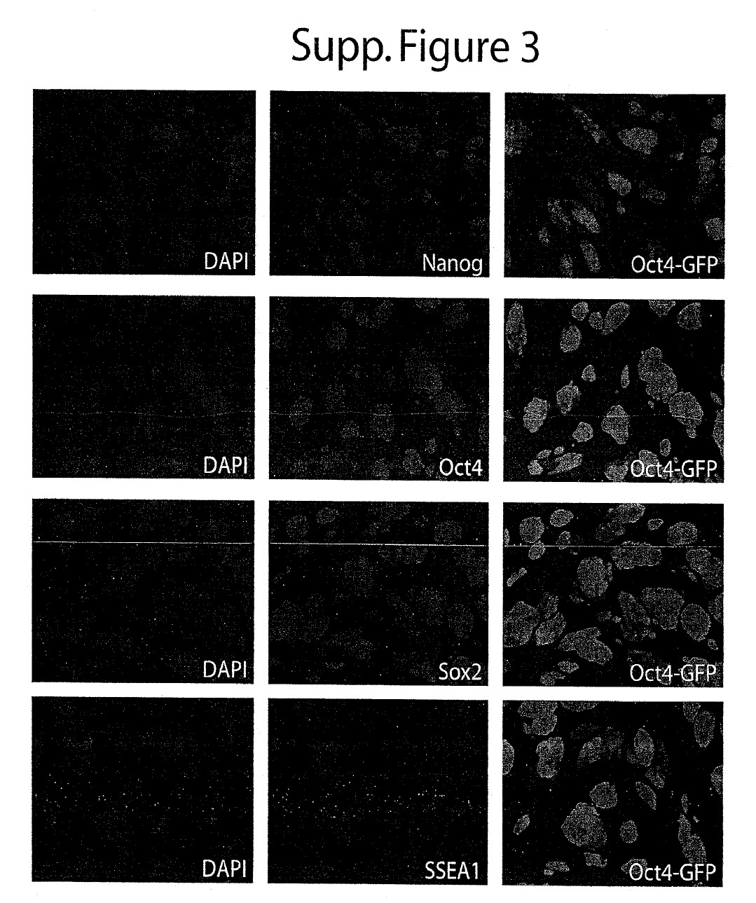

Supplementary FIG. 3. Characterization of mouse iPS-m cells.

Expression of pluripotent markers, Nanog, Oct4, Sox2 and SSEA1 were shown together with expression of Oct4-GFP in mouse iPS-m cells.

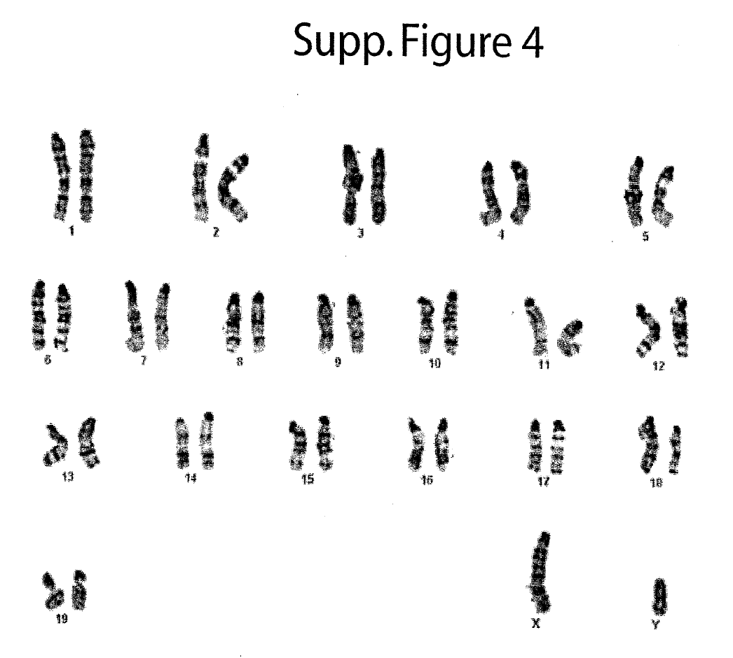

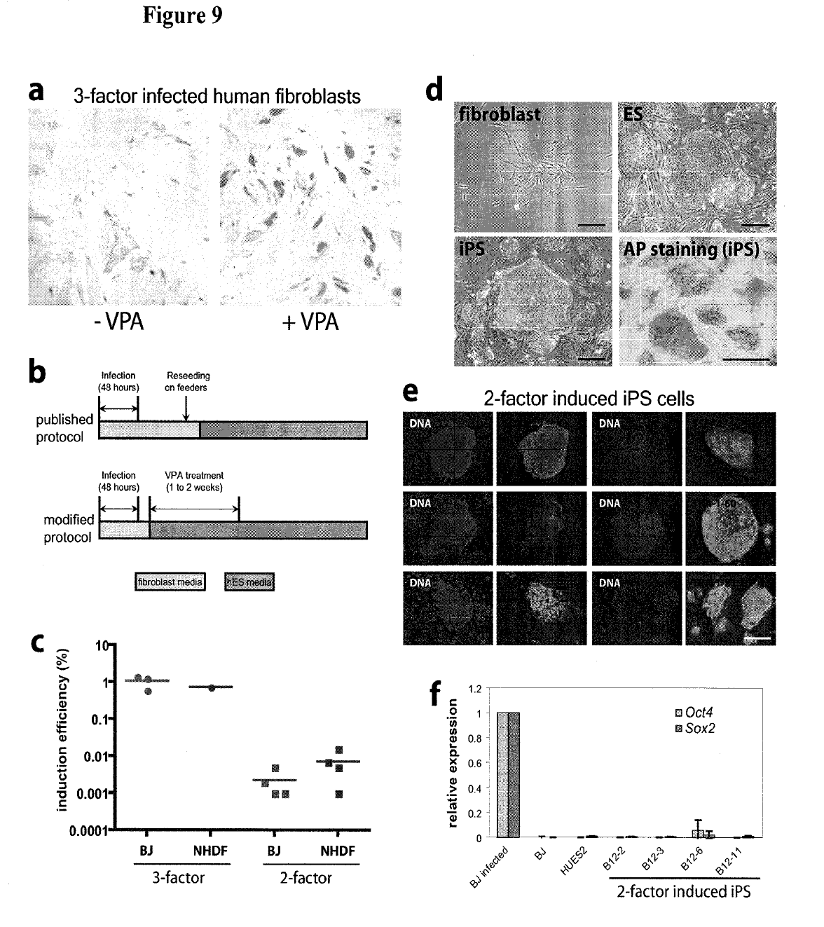

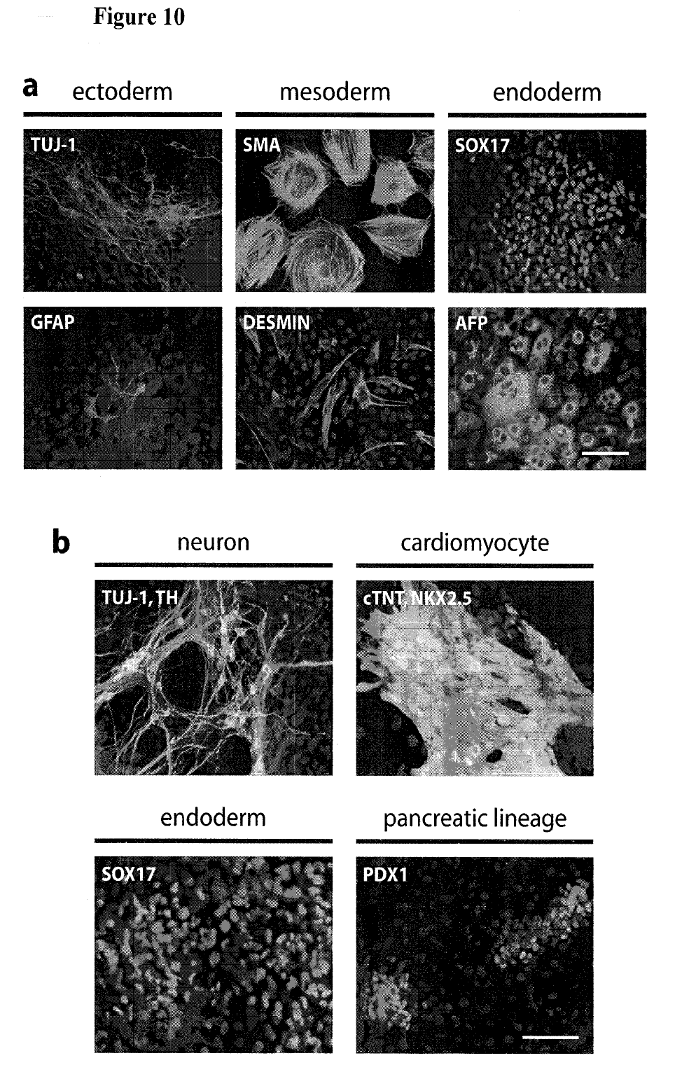

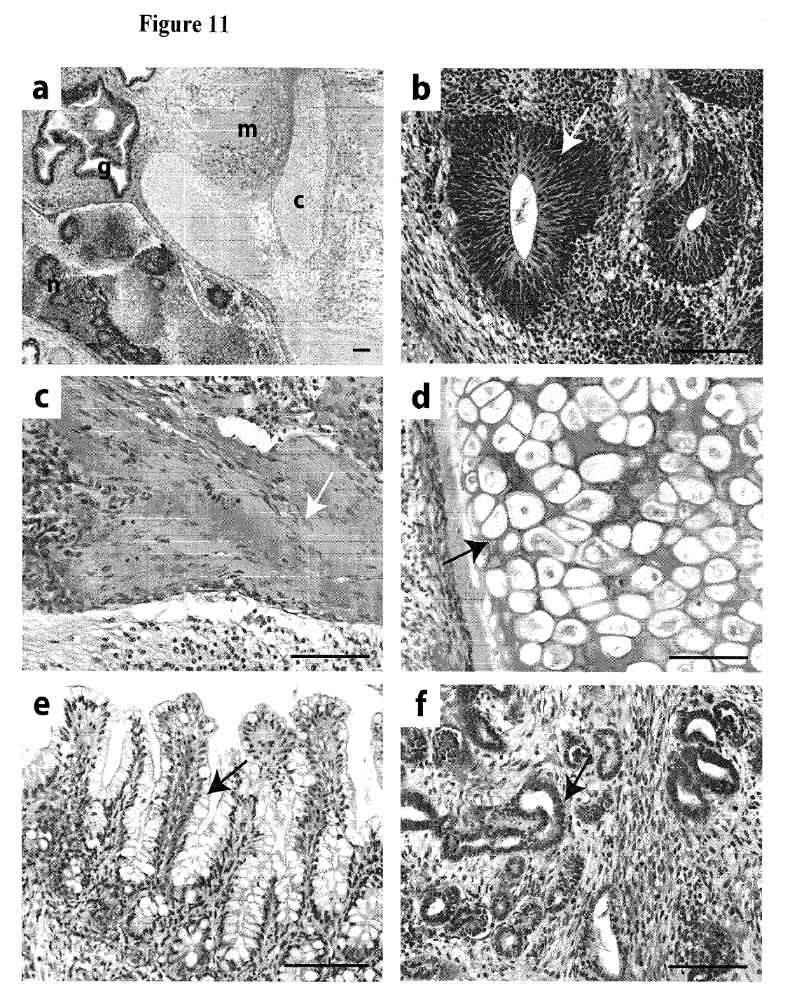

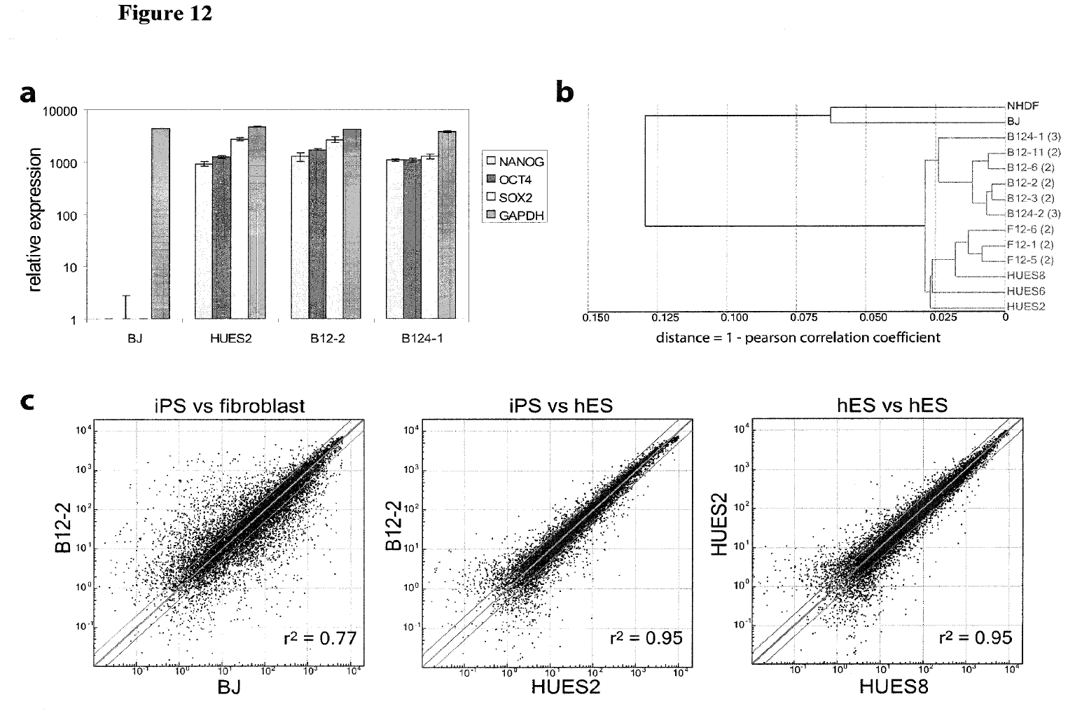

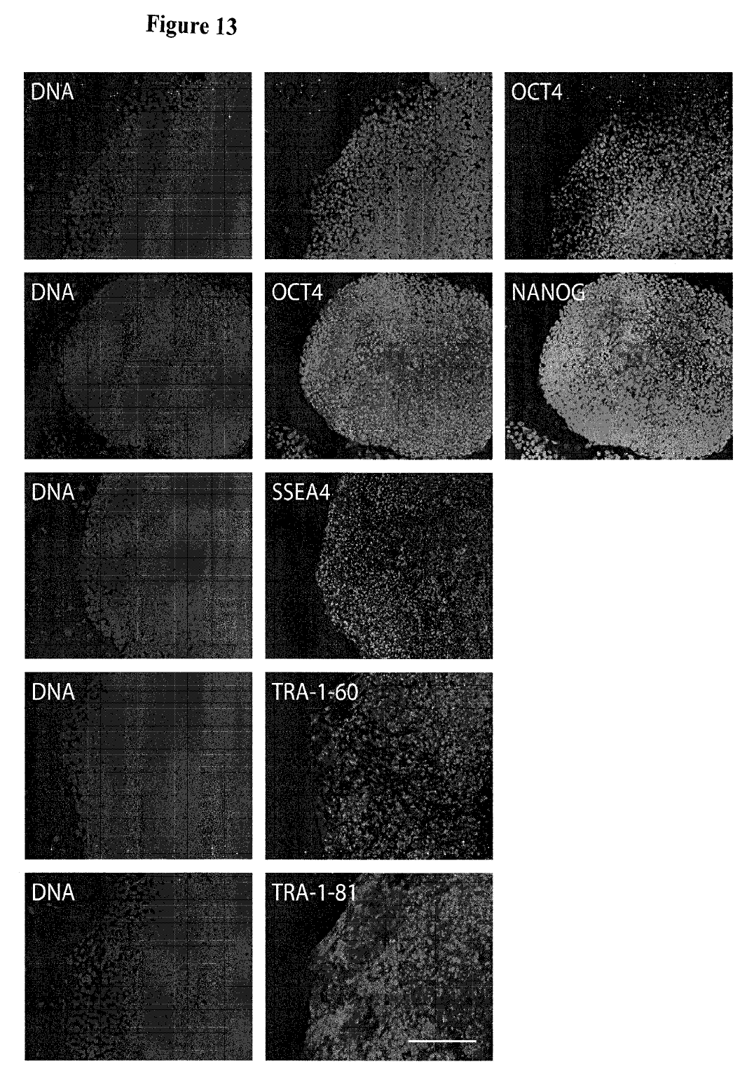

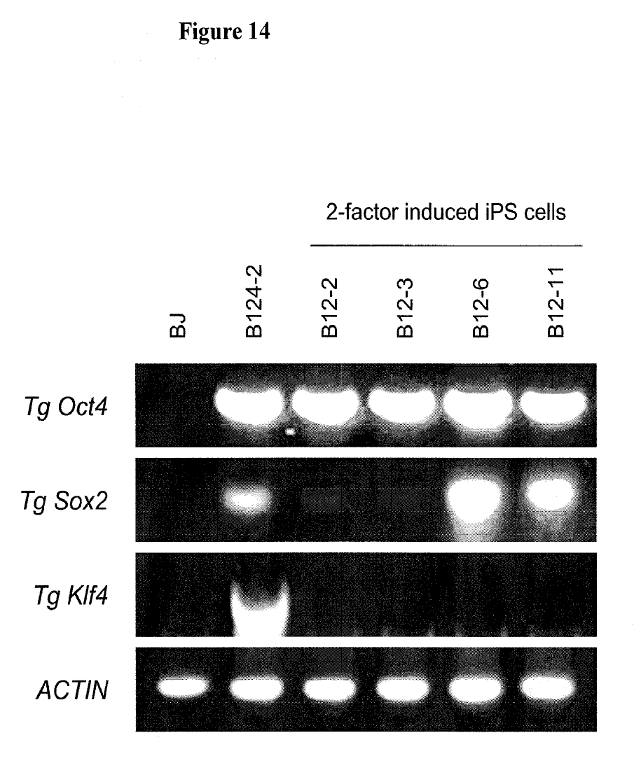

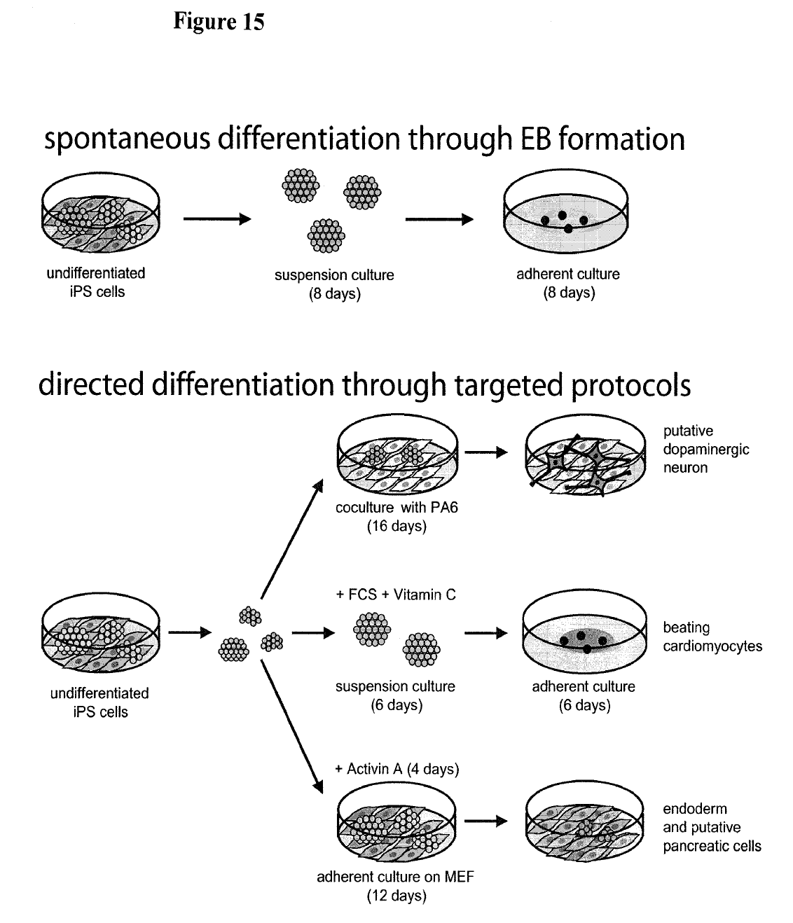

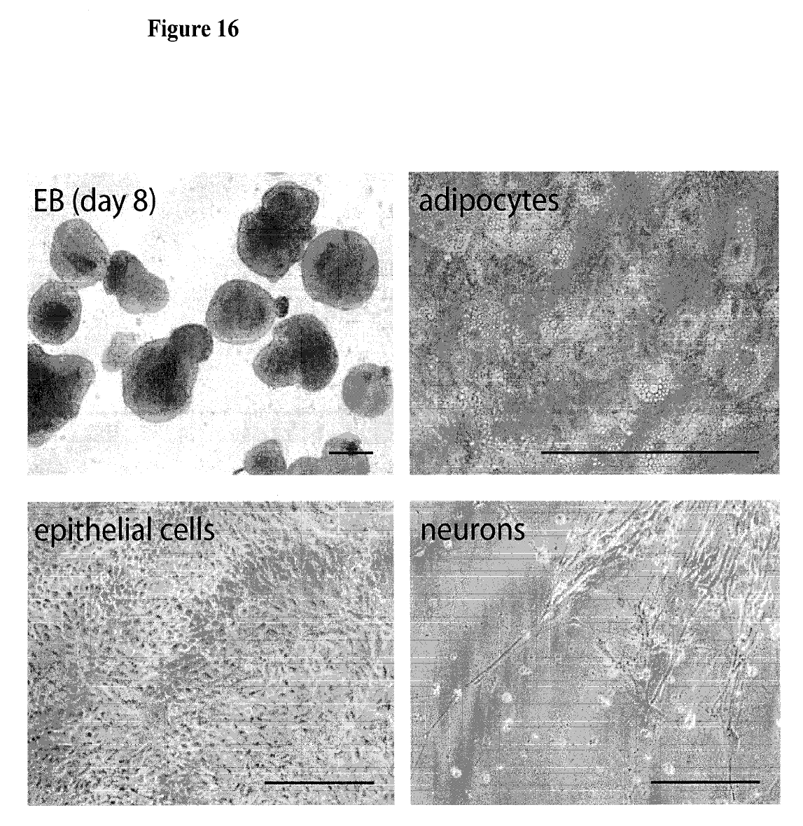

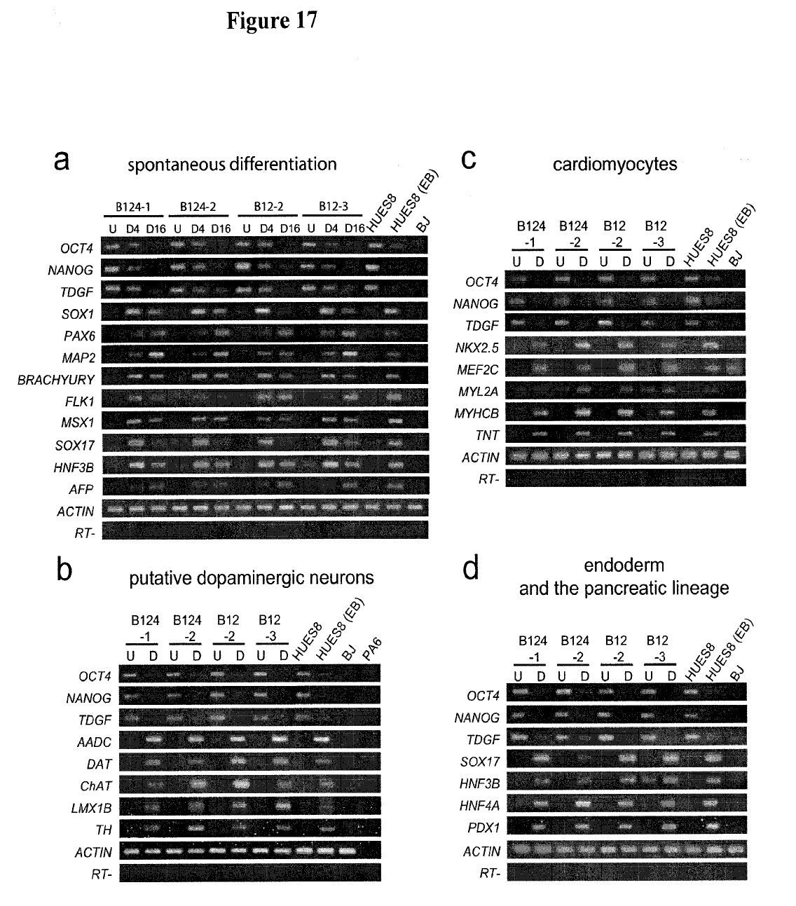

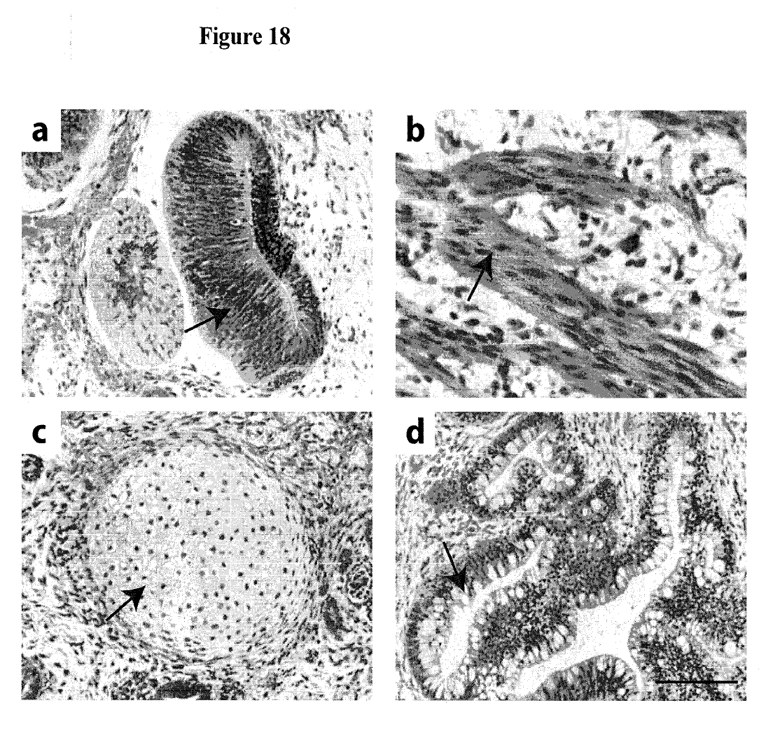

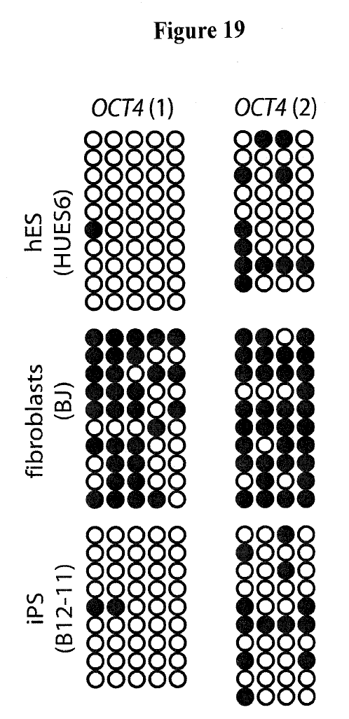

Supplementary FIG. 4. Chromosome analysis of iPS-m cells. Three out of four iPS-m cell lines (iPS-m81, 82, 83 and 84) induced by VPA treatment had normal karyotypes. Shown here is a representative chromosome analysis image from iPS-m84.

FIG. 9. VPA treatment enables induction of iPS cells with only Oct4 and Sox2

a. AP⁺ colonies were compared for 3-factor (Oct4, Sox2 and Klf4) infected BJ fibroblasts with or without VPA treatment at 25 days post-infection.

b. In the published reprogramming protocol, infected human fibroblasts are typically cultured in fibroblast media first, then reseeded on feeders and switched to hES cell media about a week post-infection. In the modified protocol, human fibroblasts were replated immediately after infection, treated with VPA in hES media, and subsequently cultured in hES media.

c. The efficiency of iPS colony induction by 3 factors (Oct4, Sox2 and Klf4) and 2 factors (Oct4 and Sox2) with VPA treatment are plotted. Each dot represents one experiment; each bar represents the average for each condition.

d. Morphology of 2-factor induced human iPS lines, as compared to BJ and hES cells, with AP staining on 2-factor induced iPS cells at the lower right corner.

e. immunofluorescence staining showed expression of pluripotent markers in 2-factor induced iPS cells.

f. Expression levels of transgenes Oct4 and Sox2, assessed by qRT-PCR, shown relative to GAPDH in 2-factor induced iPS cells, with uninfected BJ and HUES2 cells¹⁶ as negative controls. The values from the infected BJ fibroblasts were set to 1. Scale bar=250 μm.

FIG. 10. in vitro differentiation of 2-factor induced iPS cells

a. Immunofluorescence staining showed differentiation of 2-factor induced iPS cells into cells expressing markers characteristic of the three germ layers.

b. Immunofluorescence staining showed differentiation of 2-factor induced iPS cells to putative dopaminergic neurons (co-expression of TUJ-1 in green and TH (tyrosine hydroxylase) in red), cardiomyocytes (co-expression of cTNT (cardiac troponin) in green and NKX2.5 in red), definitive endoderm (50X17) and pancreatic cells (PDX1). Scale bar=100 μm.

FIG. 11. teratomas from 2-factor induced iPS cells

a. Hematoxylin and eosin staining showed the teratoma from 2-factor induced iPS cells (B12-2) contained multiple tissues, including neural (“n”), muscle (“m”), cartilage (“c”) and glandular structures (“g”). Similar results were observed for all 2-factor induced iPS cell lines examined.

b-f. higher magnification pictures showing the presence of neural epithelium (b), muscle (c) and cartilage (d), and glandular structures (e, f), indicated by arrows. Scale bar=100 μm.

FIG. 12. The gene expression profile for 2-factor induced iPS cells closely resemble that of hES cells

a. Graph showing the relative expression of OCT4, NANOG, SOX2 and GAPDH in BJ fibroblasts, hES cells (HUES2¹⁶), 2-factor induced (B 12-2) and 3-factor induced (B124-1) iPS cells.

b. Hierarchical cluster analysis of the microarray data from hES cells¹⁶, fibroblasts and iPS cells. The numbers in parenthesis indicate the number of transcription factors used for the induction of different iPS lines.

c. Scatter plots comparing global gene expression profiles between 2-factor induced iPS cells and fibroblasts, 2-factor induced iPS cells and hES cells, and two different hES cell lines. The red lines indicate the linear equivalent and two-fold differences on either side in gene expression levels.

FIG. 13. Expression of pluripotent markers in 3-factor induced iPS cells.

Immunofluorescence staining showed expression of pluripotent markers (OCT4, SOX2, NANOG, SSEA4, TRA-1-60 and TRA-1-81) in 3-factor induced iPS cells. Scale bar=250 μm.

FIG. 14. Detection of viral transgene integration in iPS cells. PCR using transgene specific primers detected integration of Oct4 and Sox2 in 2-factor induced iPS cells (B12-2, B12-3, B12-6, B12-11), and integration of Oct4, Sox2 and Klf4 in 3-factor induced iPS cells (B124-2). ACTIN was used as a internal control.

FIG. 15. Schematic drawings of in vitro differentiation of iPS cells.

For sponatenous differentiation, embryoid bodies (EBs) were generated from human iPS cells by 8 days in suspension culture. The EBs were transferred to gelatin-coated plates and cultured for another 8 days to allow further differentiation. Coculture with PA6 (stromal cells derived from skull bone marrow) was used for the differentiation into putative dopaminergic neurons as described previously^{S1}. PA6 cells were plated on gelatin-coated 6-well plates and incubated to reach confluence. Small clumps of human iPS cells were cultured for 16 days on PA6 feeder layer. The induction of cardiomyocytes was carried out as described previously^{S2}. EBs were generated from human iPS cells by 6 days in suspension culture in media containing 20% FCS and 50 mg/mL vitamin C. EBs were transferred onto gelatin-coated plates and cultured for an additional 6 days. The protocol for differentiation towards endoderm was described previously^{S3}. Briefly, undifferentiated iPS cells at approximately 80% confluence were induced to differentiate with 100 ng/ml recombinant activin A treatment for 4 days. For further induction of pancreatic-lineage cells, cells at day 4 activin A treatment were cultured for another 8 days without activin A.

FIG. 16. in vitro differentiation of 2-factor induced iPS cells through EB formation.

2-factor induced iPS cells form EBs in suspension culture. Different cell types including adipocyte, epithelial cells and neurons can be identified by morphology after EBs were allowed to differentiate further in adherent culture. Scale bar=250 μm.

FIG. 17. 2-factor induced iPS cells differentiate into derivatives of three germ layers in vitro.

a. RT-PCR analysis of pluripotent markers and various differentiation markers for the three germ layers in iPS cells that were undifferentiated (U), after 4 days in suspension culture (D4), and after 8 days in suspension culture followed by 8 days in adherent culture (D16). Two 2-factor induced iPS lines (B12-2 and B12-3) and two 3-factor induced iPS lines (B 124-1 and B 124-2) were examined. Undifferentiated hES (HUES84) cells, differentiated HUES8 cells after 16 days of EB culture, human BJ fibroblasts were used as controls.

b-d. RT-PCR analysis of pluripotent markers and differentiation markers of dopaminergic neurons (b), cardiomyocytes (c), endoderm and the pancreatic lineage (d) in iPS cells that were undifferentiated (U) and induced to differentiate into various lineages (D) through targeted protocols. Undifferentiated HUES8, differentiated HUES8 after 16 days of EB culture, human BJ fibroblasts and PA6 cells (feeder cells for differentiation of iPS cells into putative dopaminergic neurons) were used as controls.

FIG. 18. 3-factor induced iPS cells contribute to various tissues in teratomas

Hematoxylin and eosin staining of a teratoma derived from 3-factor induced iPS cells (B124-2) shows the presence of multiple tissues, including neural epithelium (a), muscle (b), cartilage (c) and glandular structures (d), indicated by arrows. Scale bar=100 μm.

FIG. 19. Methylation status of OCT4 promoter regions in 2-factor induced iPS cells.

The methylation status of the OCT4 promoter region in hES (HUES6^{S4}) cells, fibroblasts (BJ) and 2-factor induced iPS cells (B12-11, derived from BJ fibroblasts). Each horizontal row of circles represents an individual sequencing result from one amplicon. Open and filled circles indicate unmethylated and methylated CpG dinucleotides, respectively.

### DETAILED DESCRIPTION

As described herein, small molecule compounds such as VPA can be employed to efficiently generate induced pluripotent stem (iPS) cells from skin or other cell types.

iPS cells can be created by over-expression of one or more genes, for example one or more of the following four genes: Oct4, Sox2, c-Myc and Klf4 through retroviral infection, but with low efficiencies. All four of these genes are known to be or considered to be DNA binding proteins, transcription factors. Notably, the oncogene c-Myc used in this approach causes tumor formation in cells derived from the iPS cells. Although iPS cells can be generated with only Oct4, Sox2 and Klf4, the efficiency is even lower; fewer than 1 iPS colonies form out of 100,000 cells. These issues pose significant barriers for creation of iPS cells for therapeutic applications. Two obvious concerns are the use of retroviruses which integrate into chromosomal DNA and can cause ancillary problems (mutations). Beyond the use of retroviral vectors and the insertional mutations they cause, the methods involves adding new genes to the cell.

As described herein, small molecules such as VPA can improve the efficiency of iPS cell induction up to more than 100 fold. For example, treatment with 3-6 μM of 5′-azacytidine, a DNA methyltransferase inhibitor, induced 6-8% iPS cells in mouse fibroblasts infected with the four factors (Oct4, Sox2, c-Myc and Klf4), a more than 100 fold improvement over the non-treated control (˜0.04%). Three histone deacetylase inhibitors, suberoylanilide hydroxamic acid (SAHA), trichostatin A (TSA) and valproic acid (VPA), also dramatically promoted the efficiency of iPS cell creation, with VPA being the most effective among the three. Treatment with 2 mM VPA induced ˜12% iPS cells in mouse fibroblasts infected with the four factors, a more than 100 fold improvement over the non-treated control. In addition, VPA treatment induced more than 2% iPS cells in the mouse fibroblasts infected with the three factors (Oct4, Sox2 and Klf4, but not c-Myc). This effect is conserved in humans. VPA treatment promoted the efficiency of iPS colony formation by ˜30 fold in human skin cells infected with the three factors. Further optimization of the induction protocol together with VPA treatment enabled a 3-factor reprogramming efficiency of ˜1%, a significant improvement (1000 fold) over the first report on reprogramming by the same 3-factor combination (<0.001%).

As described herein, VPA treatment enables reprogramming of human cells by only 2 transcription factors, Oct4 and Sox2, without the need for the oncogenes c-Myc or Klf4. For example, iPS colonies were identified about 1 month post-infection in human fibroblasts (BJ and NHDF) infected by Oct4 and Sox2 together with VPA treatment. On average, between 1 and 5 iPS lines were successfully established out of every 100,000 BJ or NHDF cells infected by Oct4 and Sox2. Thus, the 2-factor reprogramming efficiency by VPA treatment is comparable to the induction rate for human fibroblasts infected by 3 factors (OCT4, SOX2 and KLF4), indicating VPA treatment effectively replaced the need for Klf4 and c-Myc.

The methods described herein improve the efficiency of creating iPS cells from skin (e.g., human skin cells) and are useful for making induced stem cells from other cell types without using the oncogenes c-Myc or Klf4. For example, these chemicals may make it possible to create iPS cells from small numbers of cells (e.g., such as those obtained from hair follicle cells from patients, blood samples, adipose biopsy, etc), something that could otherwise be difficult or impossible due to the low efficiency of the current method. Thus, the addition of small molecules compounds (e.g., chemicals) can increase the probability of success when trying to make iPS cells from human skin biopsies (fibroblasts or other nucleated cells) and may be helpful in creating iPS cells from any other cell types.

Stem Cells

Stem cells are cells that retain the ability to renew themselves through mitotic cell division and can differentiate into a diverse range of specialized cell types. The two broad types of mammalian stem cells are: embryonic stem cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

Stem cells can be used, e.g., in bone marrow transplants to treat leukemia. Stem cells can be used to treat diseases including cancer, Parkinson's disease, muscle damage, burns, heart disease, diabetes, osteoarthritis, rheumatoid arthritis, hematopoietic conditions (e.g., sickle cell anemia, leukemia, lymphoma, inherited blood disorders), immune deficiencies), cardiac disorders (e.g., myocardial infarcts, and myopathies) and disorders such as liver disease, diabetes, thyroid abnormalities, neurodegenerative/neurological disorders (e.g., Parkinson's Disease, Alzheimer's Disease, stroke injuries, spinal chord injuries), Crohn's Disease, circulatory disorders, respiratory disorders, wound healing and/or repair, bone repair, and enzyme abnormalities.

Cell Types for Use in the Preparation of Stem Cells

The methods described herein can be used, e.g., to reprogram somatic cells to a pluripotent state. Such somatic cells can be obtained, for example from a patient, to prepare patient-specific stem cells (e.g., patient-specific pluripotent stem cells). A variety of cells can be used, such as, hair follicle cells, a cell from a blood sample, a cell from adipose tissue, a stomach cell, a liver cell, or a cell from skin (e.g., fibroblast or other cell type, e.g., keratinocyte, melanocyte, Langerhans cell, or Merkel cell).

Somatic cells are any cells forming the body of an organism, as opposed to germline cells. In mammals, germline cells (also known as gametes) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell. For example, internal organs, skin, bones, blood, and connective tissue are all made up of somatic cells.

Additional cell types include: a fibroblast (e.g., aprimary fibroblast), a muscle cell (e.g., a myocyte), a cumulus cell, a neural cell, a mammary cell, a hepatocyte and a pancreatic islet cell. In some embodiments, the somatic cell is a primary cell line or is the progeny of a primary or secondary cell line. In one embodiment, the somatic cell is obtained from a sample, e.g., a hair follicle, a blood sample, a biopsy (e.g., a skin biopsy or an adipose biopsy), a swab sample (e.g., an oral swab sample).

Histone Deacetylase Inhibitors

Histone deacetylases (HDAC) are a class of enzymes that remove acetyl groups from an ε-N-acetyl lysine amino acid on a histone. Exemplary HDACs include those Class I HDAC: HDAC1, HDAC2, HDAC3, HDAC8; and Class II HDACs: HDAC4, HDAC5, HDAC6, HDAC7A, HDAC9, HDAC10. Type I mammalian HDACs include: HDAC1, HDAC2, HDAC3, HDAC8, and HDAC11. Type II mammalian HDACs include: HDAC4, HDAC5, HDAC6, HDAC7, HDAC9, and HDAC1.

A number of structural classes of negative regulators of HDACs (e.g., HDAC inhibitors) have been developed, for example, small molecular weight carboxylates (e.g., less than about 250 amu), hydroxamic acids, benzamides, epoxyketones, cyclic peptides, and hybrid molecules. (See, for example, Drummond D C, Noble C O, Kirpotin D B, Guo Z, Scott G K, et al. (2005) Clinical development of histone deacetylase inhibitors as anticancer agents. Annu Rev Pharmacol Toxicol 45: 495-528, (including specific examples therein) which is hereby incorporated by reference in its entirety). Non-limiting examples of negative regulators of type I/II HDACs include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (i.e., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-Cl-UCHA (i.e., 6-(3-chlorophenylureido)caproic hydroxamic acid), AOE (2-amino-8-oxo-9,10-epoxydecanoic acid), CHAP31 and CHAP 50. Other inhibitors include, for example, dominant negative forms of the HDACs (e.g., catalytically inactive forms) siRNA inhibitors of the HDACs, and antibodies that specifically bind to the HDACs. Inhibitors are available, e.g., from BIOMOL International, Fukasawa, Merck Biosciences, Novartis, Gloucester Pharmaceuticals, Aton Pharma, Titan Pharmaceuticals, Schering A G, Pharmion, MethylGene, and Sigma Aldrich. In some embodiments, VPA is a preferred histone deacetylase inhibitor.

DNA Methyltransferase Inhibitors

DNA methylation is one of the most prevalent epigenetic modifications of DNA in mammalian genomes. It is achieved by DNA methyltransferases that catalyze the addition of a methyl group from S-adenosyl-L-methionine to the 5-carbon position of cytosine. Methylation at cytosine plays an important role in regulating transcription and chromatin structure. Three families of DNA methyltransferase genes have been identified in mammals. They include Dnmt1, Dnmt2 and Dnmt3. Dnmt1 is constitutively expressed in proliferating cells and its inactivation results in demethylation of genomic DNA and embryonic death. Dnmt2 is expressed at low levels in adult tissues. Its inactivation does not affect DNA methylation or maintenance of methylation. The Dnmt3 (Dnmt3a and Dnmt3b) is strongly expressed in embryonic stem cells, but is down-regulated in differentiating embryonic stem cells and in adult somatic cells.

Most mammalian transcription factors bind GC-rich DNA elements. Methylation of these elements abolishes binding. CpG methylation is shown to induce histone deacetylation, chromatin remodeling, and gene silencing through a transcription repressor complex. CpG islands are often located around the promoters of housekeeping genes and are not methylated. In contrast, the CG sequences in inactive genes are usually methylated to suppress their expression.

Examples of nucleoside DNA methyltransferase inhibitors include 5-deoxy-azacytidine (DAC), 5-azacytidine (5-aza-CR) (Vidaza), 5-aza-2′-deoxycytidine (5-aza-CdR; decitabine), 1-B-D-arabinofuranosyl-5-azacytosine, dihydro-5-azacytidine, zebularine, Sinefungin (e.g., InSolution™ Sinefungin), 5-fluoro-2′-deoxycyticine (FdCyd). Examples of non-nucleoside DNA methyltransferse inhibitors (e.g., other than procaine) include: (−)-epigallocatechin-3-gallate (EGCG), RG108, hydralazine, procainamide, 1513-DMIa and 1513-DMIb which were isolated from the culture filtrate of Streptomyces sp. strain No. 1513, psammaplin, dominant negative forms of the DNA methyltransferases (e.g., catalytically inactive forms), oligonucleotides (e.g., including hairpin loops and specific antisense oligonucleotides (such as MG98)), siRNA inhibitors of the DNA methyltransferases, and antibodies that specifically bind to the DNA methyltransferases. Inhibitors are available, e.g., from Merck Biosciences.

Kits

The small molecules (e.g., a HDAC inhibitor(s) such as VPA and/or a DNA methyltransferase inhibitor(s)) described herein can be provided in a kit. The kit includes (a) the compounds described herein, e.g., a composition(s) that includes a compound(s) described herein, and, optionally (b) informational material. The informational material can be descriptive, instructional, marketing or other material that relates to the methods described herein and/or the use of a compound(s) described herein for the methods described herein.

The informational material of the kits is not limited in its form. In one embodiment, the informational material can include information about production of the compound, molecular weight of the compound, concentration, date of expiration, batch or production site information, and so forth. In one embodiment, the informational material relates to methods for administering the compound.

In one embodiment, the informational material can include instructions to administer a compound(s) (e.g., a HDAC inhibitor(s) such as VPA and/or a DNA methyltransferase inhibitor(s)) described herein in a suitable manner to perform the methods described herein, e.g., in a suitable dose, dosage form, or mode of administration (e.g., a dose, dosage form, or mode of administration described herein) (e.g., to a cell in vitro or a cell in vivo). In another embodiment, the informational material can include instructions to administer a compound(s) described herein to a suitable subject, e.g., a human, e.g., a human having or at risk for a disorder described herein or to a cell in vitro.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In addition to a compound(s) described herein, the composition of the kit can include other ingredients, such as a solvent or buffer, a stabilizer, a preservative, a flavoring agent (e.g., a bitter antagonist or a sweetener), a fragrance or other cosmetic ingredient, and/or an additional agent, e.g., for inducing pluripotent stem cells (e.g., in vitro) or for treating a condition or disorder described herein. Alternatively, the other ingredients can be included in the kit, but in different compositions or containers than a compound described herein. In such embodiments, the kit can include instructions for admixing a compound(s) described herein and the other ingredients, or for using a compound(s) described herein together with the other ingredients, e.g., instructions on combining the two agents prior to administration.

A compound(s) described herein can be provided in any form, e.g., liquid, dried or lyophilized form. It is preferred that a compound(s) described herein be substantially pure and/or sterile. When a compound(s) d described herein is provided in a liquid solution, the liquid solution preferably is an aqueous solution, with a sterile aqueous solution being preferred. When a compound(s) described herein is provided as a dried form, reconstitution generally is by the addition of a suitable solvent. The solvent, e.g., sterile water or buffer, can optionally be provided in the kit.

The kit can include one or more containers for the composition containing a compound(s) described herein. In some embodiments, the kit contains separate containers (e.g., two separate containers for the two agents), dividers or compartments for the composition(s) and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

### EXAMPLES

### Example 1

Patient specific stem cells can be created by reprogramming somatic cells to a pluripotent state. Recently, reprogramming of both mouse and human somatic cells was achieved by ectopic expression of specific gene combinations¹⁻⁷, however, the low efficiencies of the current methods and the introduction of exogenous genes through viral infections pose significant limitations for therapeutic applications. We report small molecule compounds that greatly improve reprogramming efficiency on fibroblasts ectopically expressing Oct4, Sox2, Klf4 and c-Myc Inhibition of DNA methyltransferase or histone deacetylase (HDAC) both greatly improves reprogramming efficiency. Treatment with valproic acid (VPA), an HDAC inhibitor, induces pluripotent stem cells efficiently without introduction of the oncogene c-Myc. VPA treatment alone partially reprograms uninfected fibroblasts by up-regulating genes specifically expressed in embryonic stem cells, and down-regulating genes specifically expressed in fibroblasts. These findings represent a first step toward reprogramming somatic cells by chemical means and provide a direct link between chromatin modification and reprogramming

Reprogramming somatic cells to a pluripotent state is traditionally achieved through somatic cell nuclear transfer (SCNT), first in frogs⁸ and then Dolly, the first cloned mammal, a decade ago⁹. Recently, SCNT has been successfully applied to primates¹⁰, suggesting reprogramming of human somatic cells may be achieved through similar methods. However, shortage of human oocytes as well as ethical and political controversies have so far thwarted progress on SCNT in human In addition, because SCNT is technically demanding and difficult to scale up, this approach is likely to be of limited use for disease therapies or mechanistic studies on reprogramming Pioneered by Yamanaka and colleagues, reprogramming by genetic means has opened a new door on somatic cell reprogramming¹⁻⁷. The forced expression of just four transcription factors, Oct4, Klf4, Sox2 and c-Myc, reprograms mouse embryonic fibroblasts (MEFs) into induced pluripotent stem (iPS) cells that closely resemble ES cells²⁻⁴. Reprogramming human somatic cells has now been achieved through similar means⁵⁻⁷, suggesting the mechanism of reprogramming is conserved between human and the mouse. However, reprogramming by viral infection is a slow and inefficient process. In addition, as has been noted by many, the genetic transformation with exogenous genes, in particular, the oncogenes such as c-Myc and Klf4^{11, 12} and the use of viral delivery systems handicap this method in terms of human therapeutic applications. Although it is now possible to make iPS cells with three factors (Oct4, Klf4, Sox2, but no c-Myc), the reprogramming process appears to take three weeks or more and fewer than 1 iPS colonies arise from 100,000 infected human fibroblasts¹³. A possible solution to these issues is to trigger the reprogramming of somatic cells using pure chemicals.

As a first step towards chemical reprogramming, we screened for small molecule compounds that improve reprogramming efficiency on MEFs infected with retroviruses expressing Oct4, Sox2, Klf4 and c-Myc. To quantitate reprogramming efficiency, we established an assay based on Fluorescence-Activated Cell Sorting (FACS) analysis using an Oct4-GFP transgenic reporter, where the expression of the green fluorescent protein (GFP) is controlled by the promoter and enhancers of Oct4, a pluripotent marker gene¹⁴. Retroviral expression of Oct4, Sox2, Klf4 and c-Myc in MEFs hemizygous for the Oct4-GFP transgene (Oct4-GFP/+) induced GFP positive cells starting at 7 days post-infection, and the percentage of GFP positive cells remained at about 0.04% between 7 and 13 days post-infection (Supplementary FIG. 1a, b and data not shown). The GFP positive cells develop into ES-like colonies expressing alkaline phosphatase at two to three weeks post-infection (Supplementary FIG. 1c, d) which can be picked and expanded as iPS cell lines (Supplementary FIG. 1e). Based on the transduction rate of 60-80% using a GFP vector, the frequency of MEFs infected with all four factors is estimated to be 13-41%. Thus, 0.1-0.3% of the 4-factor infected fibroblasts was reprogrammed, comparable to previous studies^{2-4, 15}.

We hypothesized that the induction of the pluripotent state could be facilitated by chemicals and growth factors important for the maintenance of pluripotency, because Oct4 and Sox2 are both part of the core transcriptional regulatory circuitry that controls the pluripotency of ES cells¹⁶. Both BMP and Wnt signaling are essential for the maintenance of pluripotency of mouse ES cells^{17, 18}. Treatment of 4-factor infected MEFs with BMP4 (100 ng/ml), however, had no effect on reprogramming efficiency. Activation of the Wnt pathway, using either recombinant Wnt3a (100 ng/ml) or BIO-Acetoxime (2 μM), a GSK3 inhibitor, also had no significant effect. Likewise, although inhibition of MEK and activation of protein kinase A have both been implicated in the maintenance of the pluripotent state in mouse ES cells¹⁹⁻²¹, no significant effects were observed for U0126 (2 μM) and PD98059 (40 μM), two MEK inhibitors, and forskolin (10 μM), an adenylate cyclase agonist. Thus, the mechanisms are distinct between the induction and maintenance of the pluripotent state.

We next tested whether small molecules involved in chromatin modification have any effect on reprogramming Treating 4-factor infected MEFs with 2 μM 5′-azacytidine (5′-azaC), a DNA methyltransferase inhibitor²², increased the percentage of GFP positive cells by ˜10 fold to 0.503%±0.062% (mean±standard deviation) (FIG. 1a, b). 5′-azaC promoted reprogramming efficiency in a dose-dependant manner, with an EC₅₀ of 2.4 μM (Supplementary FIG. 2a). Dexamethasone (1 μM), a synthetic glucocorticoid, improved the effect of 5′-azaC by 2.6 fold when used in combination, although dexamethasone alone had no significant effect (FIG. 1a). Three known HDAC inhibitors, suberoylanilide hydroxamic acid (SAHA), trichostatin A (TSA) and valproic acid (VPA)^{22, 23} also greatly improved reprogramming efficiency (FIG. 1a). Treatment with SAHA (5 μM) induced approximately 0.198% (±0.102%) GFP positive cells, a 10 fold improvement over the control DMSO treatment (0.018%±0.019%); and TSA treatment (20 nM) induced approximately 1.535% (±0.618%) GFP positive cells. VPA was the most potent among the three. Treating 4-factor infected MEFs with 2 mM VPA for a week induced approximately 11.8%±2.2% GFP positive cells, more than 100 fold improvement over the control (FIG. 1a, b). The reprogramming efficiency approached the estimated 13-41% viral co-transduction rate, arguing that most if not all cells infected with all four factors can be reprogrammed VPA promoted reprogramming efficiency in a dose-dependant manner, with an EC₅₀ of 1.9 mM (Supplementary FIG. 2b).

Consistent with the FACS data, GFP positive iPS colonies emerge sooner and in greater numbers with VPA treatment. At 8 days post-infection, an average of 241 colonies were observed in VPA treated MEF culture (out of 270,000 cells seeded), in contrast to no GFP positive colonies without chemical treatment. GFP positive colonies only start to emerge after 10 days post-infection in untreated cells. The dramatic difference in colony numbers was maintained as more GFP positive iPS colonies emerged in both the VPA treated and non-treated MEF culture during the following days; more than 40 fold difference in colony number was observed at two weeks post-infection (FIG. 1c).

Retroviral introduction of c-Myc could cause tumorigenecity in cells derived from the iPS cells thus generated². Although reprogramming is possible with three factors (Oct4, Sox2 and Klf4) and without c-Myc, the efficiency is extremely low and the appearance of iPS colonies is significantly delayed compared to reprogramming with four factors^{13, 24}. Nakagawa et al. found that fewer than 1 iPS colony was formed from 100,000 human dermal fibroblasts infected¹³, an efficiency that can make it difficult to derive patient-specific iPS cells from a small starting population of cells. Similar low efficiency was also reported for induction of iPS cells from mouse fibroblasts without c-Myc²⁴. We tested whether treating the cells with 5′-azaC or VPA improves the efficiency of iPS colony formation without the need for c-Myc. MEFs were first infected with Oct4, Sox2 and Klf4, then treated with 5′-azaC or VPA for a week starting 1 day post-infection. FACS analysis 10 days post-infection showed that treatment with 5′-azaC (2 μM) increased reprogramming efficiency by 3 fold, a small improvement (FIG. 2a, b). Treatment with VPA (2 mM) improved reprogramming efficiency by 50 fold (FIG. 2a, b): an efficiency superior to that achieved when MEFs are infected with all four factors (without VPA treatment). Consistent with the FACS data, a 30-40 fold increase of GFP positive colonies was observed compared to control MEFs without treatment (FIG. 2c). This allowed for picking of iPS colonies within two weeks post-infection, sooner than the typical ˜30 days post-infection or later without chemical treatment^{13, 24}.

To examine whether VPA treatment changes the type of iPS cells generated, we established multiple iPS cell lines from 3-factor infected MEFs, referred to as iPS-m cells to distinguish from iPS cells generated using all four factors. iPS-m cells induced by VPA treatment are similar to ES cells and iPS-m cells induced without drug treatment. They have typical ES/iPS cell morphology (FIG. 3a), stain for alkaline phosphatase (FIG. 3b), and express pluripotent marker genes (Supplementary FIG. 3). They were readily cultured without further chemical treatment, and passaged more than 10 times, while maintaining ES cell morphology. Microarray data of mouse iPS-m lines, MEFs and mouse ES cells (cultured under the same conditions) show that iPS-m cells induced with or without VPA treatment are distinct from MEFs, and most similar to mouse ES cells with high similarities in transcriptional profiles (FIG. 3c). The linear correlation coefficient between iPS-m cells and mouse ES cells is 0.92, comparable to previous reports⁴. In contrast, the linear correlation coefficient between iPS-m cells (or mouse ES cells) and MEFs is only 0.62. Likewise, iPS-m cells induced, with or without VPA treatment, develop teratomas in three to five weeks, and differentiate into tissues representing all three germ layers (FIG. 3d).

To further evaluate the pluripotency of the iPS-m cells induced by VPA treatment, MEFs were derived from mouse embryos carrying both the Oct4-GFP transgenic allele and the Rosa26-lacZ knock-in allele. Six iPS-m cell lines were derived from these MEFs infected with Oct4, Sox2 and Klf4 (four induced with VPA treatment, and two without VPA treatment). Following injection into mouse blastocysts, the contribution of iPS-m cells to developing mouse embryos was assessed by β-galactosidase staining at embryonic day 10.5. High-contribution chimeras were obtained from all six iPS-m cell lines, with extensive contribution of the iPS-m cell derivatives to all three germ layers (FIG. 3e, FIG. 3f). Thus, the iPS-m cells induced with VPA treatment are pluripotent and contribute to chimeric mouse embryos as do mouse ES cells or iPS cells induced without chemical treatment.

We investigated the mechanism by which VPA promotes reprogramming VPA treatment on uninfected MEFs does not induce Oct4-GFP positive cells, indicating that VPA treatment alone is insufficient to reprogram MEFs. VPA treatment does not accelerate cell cycles, a mechanism suggested for c-Myc action²⁴. Nor does VPA treatment cause detectable genetic changes when examined at the level of chromosomal abnormalities (Table 1, Supplementary FIG. 4). Microarray analysis on uninfected MEF treated with 2 mM VPA for a week showed that VPA did not have a significant effect on endogenous c-Myc gene expression either. Instead, VPA treatment partially induced an ES-like transcriptional program in uninfected MEFs. Among the 968 genes (out of 18,918 total genes) up-regulated by more than ten fold in ES cells compared to untreated MEFs, 66% are up-regulated by more than two fold in VPA treated MEFs, whereas only 4.5% are down-regulated by more than two fold (FIG. 4a). For example, Rex3 and Zfp7, two genes expressed specifically in undifferentiated ES cells, but not in untreated MEFs, are up-regulated by more than twenty fold in MEFs treated with VPA (FIG. 4b). Likewise, among the 214 genes down-regulated by more than 10 fold in ES cells compared to untreated MEFs, 55% are down-regulated by more than two fold in VPA treated MEFs, whereas only 6.2% were up-regulated by more than two fold (FIG. 4a). For example, Aspn and Meox2, two genes that are specifically expressed in MEFs but not in ES cells, were both down-regulated by more than twenty fold in VPA treated MEFs (FIG. 4c). Therefore, VPA partially reprograms MEFs towards more ES cell-like.

TABLE 1
Karyotype analysis on iPS-m cell lines.

| Cell line | Karyotype (20 cells analyzed per cell line) |
|---|---|
| iPS-m81 | 40, XY[18]/80, XXYY[2] |
| iPS-m82 | 40, XY[20] |
| iPS-m83 | 43, XY, +7, +8, +12[1]/80, XXYY[3]/40, XY[15] |
| iPS-m84 | 40, XY[19] |

These findings provide insights into the mechanism of reprogramming The demonstration that DNA methyltransferase and HDAC inhibitors improve reprogramming efficiency suggests that chromatin modification is a key step in reprogramming fibroblasts to pluripotent cells. The effect of dexamethasone, a glucocorticoid that promotes transdifferentiation²⁵, suggests that reprogramming and transdifferentiation may share common mechanisms. In addition, the fact that small molecules and growth factors that promote ES cell self-renewal do not appear to increase reprogramming efficiency, suggests that reprogramming and ES/iPS cell self-renewal involve distinct mechanisms.

The identification of the small molecules reported here is a proof of principle that chemicals can increase reprogramming efficiency and replace one or more factors used for reprogramming Given that the reprogramming mechanism is highly conserved between human and the mouse, the findings will likely apply to human cells and our preliminary experiments with human fibroblasts suggest this is the case.

METHODS

Derivation of MEFs and Cell Culture

MEFs were derived from e13.5 embryos hemizygous for the Oct4-GFP transgenic allele. Embryos were sexed by inspecting gonads for the pattern of Oct4-GFP expression. Gonads and internal organs were removed before processing the embryos for MEF isolation. To generate iPS cells that can be identified in mouse chimeras after blastocyst injection, we derived MEFs from e13.5 embryos that are hemizygous for Oct4-GFP and heterozygous for the Rosa26-lacZ reporter allele. MEFs were grown in DMEM supplemented with 10% FBS, L-glutamine, penicillin/streptoMycin, nonessential amino acids, and sodium pyruvate. MEFs in early passages (up to passage 5) were used for generation of iPS cells.

Retrovirus Production and Small Molecule Screening

Moloney-based retroviral vector (pMXs) containing the murine complementary DNAs of Oct4, Sox2, c-Myc, and Klf4 were obtained from Addgene¹. These plasmids were co-transfected into 293T cells with packaging vectors (pUMVC and pCMV-VSVG), and viral supernatants were collected 48 hours post-transfection to infect MEFs. Two to three rounds of infection were performed during a 48 hour period. The first day after viral supernatants were removed was defined as 0 day post-infection. Infected MEFs were subsequently cultured in mouse ES cell media (Knockout DMEM supplemented with 15% Hyclone FBS, L-glutamine, penicillin/streptoMycin, nonessential amino acids, β-mercaptoethanol, and with 1000 U/ml LIF), and treated with small molecules or growth factors for a week starting from 1 or 2 days post-infection. After the treatment, cells were cultured in mouse ES cell media, and collected for FACS analysis typically between 9 and 11 days post-infection. All conditions were tested in quadruplicates.

Generation of iPS Cells

For the generation of mouse iPS cells, infected MEFs were cultured in mouse ES cell media until iPS colonies were ready to be picked. In some experiments, knockout serum replacement was used instead of the Hyclone FBS in the mouse ES cell media, which appeared to accelerate the reprogramming process, consistent with a recent report²⁶. Chemical treatment started 1 or 2 days post-infection and lasted for a week in general. iPS colonies were picked between 9-21 days post-infection based on GFP expression and colony morphology. The picked colonies were then expanded and maintained on irradiated MEF feeder layers in mouse ES cell media.

Generation of Teratoma and Chimeras

Teratomas were produced by injecting 1 million cells subcutaneously into NOD-SCID mice. Palpable tumors developed in 2-3 weeks. Tumor samples were collected in 5 weeks, fixed in 4% paraformaldehyde and processed for pafaffin embedding and hematoxylin and eosin staining following standard procedures.

Blastocysts were obtained through mating of hormone primed female BDF1 and male BDF1 or C57BL/6J mice. Chimeras were produced by injecting iPS cells into blastocysts, followed by implantation into pseudopregnant ICR mice. Chimeric embryos were dissected at e10.5 (8 days after injection) and analyzed for β-galatosidase activity following standard protocols. Stained embryos were then fixed and embedded in paraffin, and sections were counterstained with nuclear fast red.

Use of Chemicals and Growth Factors

The following chemicals are used: 5′-azaC from Sigma-Aldrich, SAHA from Biomol International, BIO-Acetoxime (GSK-3 Inhibitor X), dexamethasone, Forskolin, PD98059, TSA, U0126, and VPA from EMD Biosciences. Stock solutions of 5′-azaC and VPA were made in PBS or media. Stock solutions of all other chemicals were made in DMSO. We purchased recombinant mouse Wnt3a and recombinant human Bmp4 from Roche.

Alkaline Phosphatase and Immunofluorescence Staining

Alkaline phosphatase staining was performed with the Vector Red substrate kit from Vector Laboratories Immunofluorescence staining were performed using the following primary antibodies: rabbit anti-GFP (Molecular Probes), rabbit anti-mNanog (Cosmobio), mouse anti-mOct4 (Santa Cruz Biotechnology), goat anti-Sox2 (Santa Cruz Biotechnology), mouse anti-SSEA1 (Developmental Studies Hybridoma Bank)

Whole-Genome Expression Analysis

For transcriptional analysis, total RNA was isolated from cells cultured in 6 well dishes using RNeasy Mini Kit and QlAshredder from Qiagen. Biotinylated antisense RNA were amplified using Illumina Total Prep RNA amplification Kit from Ambion, hybridized to Illumina Whole-Genome Expression BeadChips (MouseRef-8) and analyzed by Illumina Beadstation 500. All samples were prepared in two to three biological repeats. Data were analyzed using the Beadstudio software provided by Illumina.

### References (for Example 1):

1. Takahashi, K. & Yamanaka, S. Induction of pluripotent stem cells from mouse embryonic and adult fibroblast cultures by defined factors. Cell 126, 663-676 (2006).
2. Okita, K., Ichisaka, T. & Yamanaka, S. Generation of germline-competent induced pluripotent stem cells. Nature 448, 313-317 (2007).
3. Wernig, M. et al. In vitro reprogramming of fibroblasts into a pluripotent ES-cell-like state. Nature 448, 318-324 (2007).
4. Maherali, N. et al. Directly Reprogrammed Fibroblasts Show Global Epigenetic Remodeling and Widespread Tissue Contribution. Cell Stem Cell 1, 55-70 (2007).
5. Takahashi, K. et al. Induction of Pluripotent Stem Cells from Adult Human Fibroblasts by Defined Factors. Cell (2007).
6. Yu, J. et al. Induced Pluripotent Stem Cell Lines Derived from Human Somatic Cells. Science (2007).
7. Park, I. H. et al. Reprogramming of human somatic cells to pluripotency with defined factors. Nature (2007).
8. Gurdon, J. B., Elsdale, T. R. & Fischberg, M. Sexually mature individuals of Xenopus laevis from the transplantation of single somatic nuclei. Nature 182, 64-65 (1958).
9. Wilmut, I., Schnieke, A. E., McWhir, J., Kind, A. J. & Campbell, K. H. Viable offspring derived from fetal and adult mammalian cells. Nature 385, 810-813 (1997).
10. Byrne, J. et al. Producing primate embryonic stem cells by somatic cell nuclear transfer. Nature (2007).
11. Hanahan, D. & Weinberg, R. A. The hallmarks of cancer. Cell 100, 57-70 (2000).
12. Rowland, B. D. & Peeper, D. S. KLF4, p21 and context-dependent opposing forces in cancer. Nat. Rev. Cancer 6, 11-23 (2006).
13. Nakagawa, M. et al. Generation of induced pluripotent stem cells without Myc from mouse and human fibroblasts. Nat. Biotechnol. (2007).
14. Szabo, P. E., Hubner, K., Scholer, H. & Mann, J. R. Allele-specific expression of imprinted genes in mouse migratory primordial germ cells. Mech. Dev. 115, 157-160 (2002).
15. Meissner, A., Wernig, M. & Jaenisch, R. Direct reprogramming of genetically unmodified fibroblasts into pluripotent stem cells. Nat. Biotechnol. 25, 1177-1181 (2007).
16. Boyer, L. A. et al. Core transcriptional regulatory circuitry in human embryonic stem cells. Cell 122, 947-956 (2005).
17. Ying, Q. L., Nichols, J., Chambers, I. & Smith, A. BMP induction of Id proteins suppresses differentiation and sustains embryonic stem cell self-renewal in collaboration with STAT3. Cell 115, 281-292 (2003).
18. Sato, N., Meijer, L., Skaltsounis, L., Greengard, P. & Brivanlou, A. H. Maintenance of pluripotency in human and mouse embryonic stem cells through activation of Wnt signaling by a pharmacological GSK-3-specific inhibitor. Nat Med 10, 55-63 (2004).
19. Burdon, T., Stracey, C., Chambers, I., Nichols, J. & Smith, A. Suppression of SHP-2 and ERK signalling promotes self-renewal of mouse embryonic stem cells. Dev Biol 210, 30-43 (1999).
20. Chen, S. et al. Self-renewal of embryonic stem cells by a small molecule. Proc. Natl. Acad. Sci. U. S. A. 103, 17266-17271 (2006).
21. Faherty, S., Fitzgerald, A., Keohan, M. & Quinlan, L. R. Self-renewal and differentiation of mouse embryonic stem cells as measured by Oct4 expression: the role of the cAMP/PKA pathway. In Vitro Cell Dev. Biol. Anim. 43, 37-47 (2007).
22. Yoo, C.B. & Jones, P.A. Epigenetic therapy of cancer: past, present and future. Nat. Rev. Drug Discov. 5, 37-50 (2006).
23. Drummond, D. C. et al. Clinical development of histone deacetylase inhibitors as anticancer agents. Annu. Rev. Pharmacol. Toxicol. 45, 495-528 (2005).
24. Wernig, M., Meissner, A., Cassady, J. P. & Jaenisch, R. C-Myc Is Dispensable for Direct Reprogramming of Mouse Fibroblasts. Cell Stem Cell (2007).
25. Slack, J. M. & Tosh, D. Transdifferentiation and metaplasia—switching cell types. Curr. Opin. Genet. Dev. 11, 581-586 (2001).
26. Blelloch, R., Venere, M., Yen, J. & Ramalho-Santos, M. Generation of Induced Pluripotent Stem Cells in the Absence of Drug Selection. Cell Stem Cell 1, 245-247 (2007).

### Example 2

Patient specific stem cells may be created by reprogramming somatic cells to a pluripotent state. Ectopic expression of defined sets of transcription factors can reprogram mouse and human somatic cells to induced pluripotent stem (iPS) cells that closely resemble embryonic stem (ES) cells¹⁻⁸. The current low reprogramming efficiency hinders mechanistic studies on reprogramming, and the viral expression of exogenous genes, in particular oncogenes c-Myc and Klf4, may handicap this method for human therapeutic applications. We found that valproic acid (VPA), a histone deacetylase inhibitor, increases the efficiency of reprogramming, allowing us to re-investigate the transcription factors required for reprogramming human somatic cells to a pluripotent state. Here we report that VPA treatment enables reprogramming by only 2 transcription factors, Oct4 and Sox2, without the need for the oncogenes c-Myc or Klf4. The 2-factor induced human iPS cells resemble human embryonic stem (hES) cells both in gene expression and pluripotency. The replacement of transcription factors with a chemical to induce pluripotent stem cells from human fibroblasts opens the door to reprogramming with pure chemicals.

Ectopic expression of the transcription factors OCT4, SOX2, KLF4 and c-MYC or a different set of 4 factors (OCT4, SOX2, NANOG and LIN28) reprograms somatic cells to a pluripotent state¹⁻⁸. More recently, it was shown that a 3-factor combination of OCT4, SOX2 and KLF4 can also reprogram mouse and human somatic cells^{9,10}. The 3-factor reprogramming efficiency is low; fewer than 1 iPS colony was formed from 100,000 (<0.001%) human fibroblasts. While the 3-factor reprogrammed human cells were shown to be pluripotent by in vitro differentiation, the absence of teratoma assays and a full analysis of their transcriptional profiles leaves open the possibility that there may be some differences between these cells and those reprogrammed with 4 factors.

We set out to explore the possibility of using chemicals to replace the need of one or more factors in the 4-factor combination (Oct4, Sox2, Klf4 and c-Myc) to reprogram human somatic cells, having recently shown that histone deacetylase (HDAC) inhibitors improve reprogramming efficiency of mouse embryonic fibroblasts (MEFs) by the 4 factors (manuscript submitted). Valproic acid (VPA), one of the HDAC inhibitors, enables efficient induction of pluripotent stem (iPS) cells from mouse fibroblasts infected by 3 factors, Oct4, Sox2 and Klf4 (˜2% based on induction of Oct4-GFP⁺ cells). We therefore examined the effect of VPA on 3-factor reprogramming of primary human fibroblasts, BJ (neonatal human foreskin fibroblasts from ATCC) and NHDF (normal human dermal fibroblasts, neonatal, from Lonza Bioscience).

Human fibroblasts were first infected by Moloney murine leukemia retroviruses expressing the Oct4, Sox2 and Klf4 genes, and treated with VPA for 1 to 2 weeks. In both BJ and NHDF, VPA increased the number of alkaline phosphatase positive (AP⁺) colonies by 10 to 30 fold when examined at about 1 month post-infection (FIG. 9a). Further optimization of the induction protocol (FIG. 9b) together with VPA treatment enabled a 3-factor reprogramming efficiency of ˜1% (FIG. 9c). This represents a significant improvement (1000 fold) over the first report on reprogramming by the same 3-factor combination (<0.001%)⁹. iPS colonies can be easily identified by their morphology, and picked and expanded to establish iPS cell lines. The 3-factor induced iPS cells closely resembled hES cells in pluripotent marker expression (FIG. 13), pluripotency and global gene expression profiles (described in more details below). Thus, with VPA treatment, human somatic cells can be reprogrammed efficiently by 3 transcription factors (Oct4, Sox2 and Klf4).

Encouraged by the more efficient 3-factor reprogramming with VPA treatment, we explored the possibility of eliminating some of the transcription factors. The modified induction method with VPA treatment (FIG. 9b) was applied to human BJ and NHDF cells infected by different 2-factor combinations, and AP⁺ iPS colonies were identified about 1 month post-infection in fibroblasts infected by Oct4 and Sox2. Karyotypically normal iPS cell lines (Table 2) were established from 2-factor infected human fibroblasts. On average, between 1 and 5 iPS lines were successfully established out of every 100,000 BJ or NHDF cells infected by Oct4 and Sox2 (FIG. 9c). Thus, the 2-factor reprogramming efficiency by VPA treatment is comparable to the published induction rate for human fibroblasts infected by 3 factors (OCT4, SOX2 and KLF4)⁹, indicating VPA treatment effectively replaced the need for Klf4 and c-Myc. The efficiency of reprogramming by 2 factors, however, was ˜100 fold lower than that by 3 factors both with VPA treatment. Therefore, KLF4, although dispensable for reprogramming, plays a facilitating role as has been described for c-MYC^{9, 10}.

TABLE 2
Karyotype analysis on 2-factor induced iPS cells

| Cell line | Karyotype |
|---|---|
| B12-2 | 46, XY[2] |
| B12-3 | 46, XY[15] |
| B12-6 | 46, XY[20] |
| B12-11 | 46, XY[17], 45, XY, −8[3] |

The 2-factor induced human iPS cells were readily cultured in standard hES culture media without further VPA treatment. DNA fingerprinting analysis (Table 3) confirmed the fibroblast-origin of the reprogrammed cells. The 2-factor induced human iPS cells were morphologically similar to hES cells, and stain positive for AP (FIG. 9d). Immunofluorescence staining confirmed expression of pluripotent markers, including NANOG, OCT4, SOX2, SSEA4, TRA-1-60 and TRA-1-81, in the 2-factor induced human iPS cells (FIG. 9e). The genomic integration of the Oct4 and Sox2 transgenes was confirmed by PCR (FIG. 14). Quantitative RT-PCR (qRT-PCR) to detect expression of the viral transgenes showed silencing of the viral Oct4 and Sox2 in the iPS lines examined, indicating that the maintenance of the iPS phenotype is independent of continued transgene expression (FIG. 9f).

TABLE 3
DNA fingerprint analysis on 2-factor induced iPS cells and
parental fibroblast lines.

| Genomic loci | BJ | B12-2 | B12-3 | NHDF | F12-5 |
|---|---|---|---|---|---|
| Amelogenin | X, Y | X, Y | X, Y | X, Y | X, Y |
| D18S51 | 17, 19 | 17, 19 | 17, 19 | 13, 16 | 13, 16 |
| vWA | 16, 18 | 16, 18 | 16, 18 | 16 | 16 |
| Penta E | 7, 17 | 7, 17 | 7, 17 | 7 | 7 |
| D8S1179 | 9, 11 | 9, 11 | 9, 11 | 11, 12 | 11, 12 |
| D5S818 | 12 | 12 | 12 | 11, 13 | 11, 13 |
| TPOX | 10, 11 | 10, 11 | 10, 11 | 8, 9 | 8, 9 |
| D13S317 | 8, 9 | 8, 9 | 8, 9 | 12 | 12 |
| FGA | 22, 23 | 22, 23 | 22, 23 | 22, 24 | 22, 24 |
| D7S820 | 11, 12 | 11, 12 | 11, 12 | 10 | 10 |
| D3S1358 | 14, 16 | 14, 16 | 14, 16 | 15, 18 | 15, 18 |
| D16S539 | 9, 13 | 9, 13 | 9, 13 | 12, 13 | 12, 13 |
| THO1 | 7, 8 | 7, 8 | 7, 8 | 8, 9.3 | 8, 9.3 |
| CSF1PO | 10, 12 | 10, 12 | 10, 12 | 10 | 10 |
| D21S11 | 29 | 29 | 29 | 29, 30 | 29, 30 |
| Penta D | 12, 13 | 12, 13 | 12, 13 | 9, 11 | 9, 11 |

Shown here are DNA fingerprint results on BJ, BJ derived 2-factor induced iPS cells (B12-2 and B12-3), NHDF, and NHDF derived 2-factor induced iPS cells (F12-5). Fifteen polymorphic short tandem repeat (STR) DNA loci plus Amelogenin for sex chromosomes were analyzed.

The ability of ES cells to differentiate into all cell types is the basis for their potential in regenerative medicine. We examined the differentiation capacity of the iPS cells in vitro (FIG. 15) and in vivo. Like hES cells, the 2-factor induced iPS cells form embryoid bodies in suspension culture (FIG. 16), some of which exhibit rhythmic beating, characteristic of contractile cardiomyocytes, a mesoderm derivative. Spontaneous differentiation of iPS cells was evident when these embryoid bodies were allowed to grow in adherent culture on gelatin-coated plates. Epithelial cells, adipocytes and neurons were identified by morphology (FIG. 16) Immunofluorescence staining and RT-PCR analysis confirmed differentiation of the 2-factor induced iPS cells into derivatives of three embryonic gem layers (FIG. 10a, FIG. 17a). We also examined whether directed differentiation of 2-factor induced iPS cells could be induced through protocols established for hES cells. 2-factor induced iPS cells were successfully differentiated into neurons co-expressing TUJ-1 and TH (tyrosine hydroxylase), the latter being a marker for dopaminergic neurons (ectoderm derivative), beating cardiomyocytes (mesoderm derivative), and definitive endoderm as well as endoderm derivative pancreatic cells following established protocols for these cell types¹¹⁻¹⁵. Expression of markers characteristic of these differentiated cell types was confirmed by immunofluorescence staining and RT-PCR analysis (FIG. 10b, Supplementary FIG. 17b-d).

Like ES cells, the 2-factor induced human iPS cells developed teratomas after subcutaneous injection into immunocompromised NOD-SCID mice. Histological examination of the teratomas revealed multiple tissues, including neural epithelium, muscle, cartilage and various glandular structures (FIG. 11). Thus, the 2-factor induced iPS cells have the capacity to differentiate both in vitro and in vivo, and appear to respond to the same signals that direct hES differentiation. Similar in vitro and in vivo differentiation experiments were performed on 3-factor induced iPS cell lines (FIG. 17, 18), and no qualitative differences were detected in the differentiation capacity between 2-factor and 3-factor induced iPS cells.

To further compare the 2-factor induced iPS cells with hES cells, we examined DNA methylation patterns and global gene expression profiles. The OCT4 promoter regions, examined by bisulphite sequencing, were demethylated in 2-factor induced iPS cells, relative to the parental fibroblast line (FIG. 19). Microarray analysis showed that mRNA expression levels for pluripotent markers genes including OCT4, NANOG, and SOX2, in both 2-factor and 3-factor induced iPS cells, were comparable to hES cells¹⁶, and were markedly elevated compared to fibroblasts (FIG. 12a). In these assays the OCT4 and SOX2 mRNA were transcribed from endogenous loci, which can be distinguished from the viral transgenes that express murine Oct4 and Sox2.

The global gene expression profiles of both 2-factor and 3-factor induced iPS cell lines closely resembled those of hES cells (FIG. 12b, c). The linear coefficient of determination (r², the square of the correlation coefficient) values between iPS cells (or hES cells) and fibroblasts were ˜0.76. In contrast, the r² values were ˜0.95 between various 2-factor induced iPS and hES lines, comparable to the r² values between different hES lines (Table 4). This indicates that the difference between iPS and hES cells is no greater than the difference between different hES cell lines. We conclude that, although there is difference in induction efficiencies, there is no significant difference between the products for 2-factor versus 3-factor induced iPS cells. In terms of their global gene expression patterns, and similarity to hES cells, induction of iPS cells with Oct4 and Sox2, plus VPA, produces oncogene-free pluripotent stem cells like those produced by Oct4, Sox2 and Klf4.

TABLE 4
coefficient of determination (r2) values between fibroblast, hES and iPS cell lines.

| | | Fibroblasts | | hES | | | 3-factor induced iPS | | 2-factor induced iPS | | | | | | |
|---|---|---|---|---|---|---|---|---|---|---|---|---|---|---|---|
| | | BJ | NHDF | HUES2 | HUES6 | HUES8 | B124-1 | B124-2 | B12-2 | B12-3 | B12-6 | B12-11 | F12-1 | F12- [symbol] | F12- [symbol] |
| fibroblast | BJ | 1 | [symbol] | [symbol] | [symbol] | [symbol] | [symbol] | [symbol] | [symbol] | [symbol] | [symbol] | [symbol] | [symbol] | [symbol] | [symbol] |
| | NHDF | | 1 | [symbol] | [symbol] | [symbol] | [symbol] | [symbol] | [symbol] | [symbol] | [symbol] | [symbol] | [symbol] | [symbol] | [symbol] |
| hES | HUES2 | | | 1 | [symbol] | [symbol] | [symbol] | [symbol] | [symbol] | [symbol] | [symbol] | [symbol] | [symbol] | [symbol] | [symbol] |
| | HUES6 | | | | 1 | [symbol] | [symbol] | [symbol] | [symbol] | [symbol] | [symbol] | [symbol] | [symbol] | [symbol] | [symbol] |
| | HUES8 | | | | | 1 | [symbol] | [symbol] | [symbol] | [symbol] | [symbol] | [symbol] | [symbol] | [symbol] | [symbol] |
| iPS cells | B124-1 | | | | | | 1 | [symbol] | [symbol] | [symbol] | [symbol] | [symbol] | [symbol] | [symbol] | [symbol] |
| from BJ | B124-2 | | | | | | | 1 | [symbol] | [symbol] | [symbol] | [symbol] | [symbol] | [symbol] | [symbol] |
| | B12-2 | | | | | | | | 1 | [symbol] | [symbol] | [symbol] | [symbol] | [symbol] | [symbol] |
| | B12-3 | | | | | | | | | 1 | [symbol] | [symbol] | [symbol] | [symbol] | [symbol] |
| | B12-6 | | | | | | | | | | 1 | [symbol] | [symbol] | [symbol] | [symbol] |
| | B12-11 | | | | | | | | | | | 1 | [symbol] | [symbol] | [symbol] |
| iPS cells | F12-1 | | | | | | | | | | | | 1 | [symbol] | [symbol] |
| from | F12- [symbol] | | | | | | | | | | | | | 1 | [symbol] |
| NHDF | F12- [symbol] | | | | | | | | | | | | | | 1 |

r² between different hES lines were highlighted in blue, and r2 between 2-factor induced iPS lines and hES lines were highlighted in red. [symbol]
indicates data missing or illegible when filed

In summary, these experiments support two conclusions. First, VPA, an HDAC inhibitor, increases reprogramming efficiency of both human and mouse fibroblasts, enabling a ˜1% reprogramming efficiency on primary human fibroblasts infected with the transcription factors Oct4, Sox2 and Klf4. This reasonably high efficiency of reprogramming may allow derivation of patient-specific iPS cells from a small starting population of cells, and may facilitate mechanistic studies of reprogramming, such as detection of early changes during the process. The effect of VPA and other HDAC inhibitors on reprogramming (manuscript submitted) suggests that chromatin remodeling is a rate limiting step in the whole process. The second conclusion begins to address concerns about the integration of viral transgenes into the somatic genome¹⁷⁻¹⁹, in particular, the oncogenes c-MYC and KLF4. Our results provide the first example of the use of a chemical to replace the need for a transcription factor for the generation of human iPS cells. The elimination of oncogenes c-MYC and KLF4 is likely to be essential for any therapeutic use of reprogrammed cells. Our results are consistent with the roles of OCT4 and SOX2 in the maintenance of pluripotency²⁰, and support a central role for OCT4 and SOX2 in the induction of a pluripotent state, consistent with OCT4 and SOX2 being the only overlapping factors required for reprogramming human somatic cells. Together, these results raise the question of whether it will be possible to find small molecules to replace OCT4 and SOX2 and achieve reprogramming through purely chemical means, making therapeutic use of reprogrammed cells safer and more practical.

Methods Summary

Cell Culture

Human BJ (ATCC CRL-2522) and NHDF (Lonza Biosciences CC-2509) cells were cultured in fibroblast medium: DMEM/M199 (4:1) supplemented with 15% FBS, L-glutamine and penicillin/streptomycin. hES and iPS cells were cultured in hES cell media: Knockout DMEM supplemented with 10% knockout serum replacement, 10% human plasma fraction, 10 ng/ml bFGF, nonessential amino acids, β-mercaptoethanol, L-glutamine, and penicillin/streptomycin.

Retrovirus Production

Moloney-based retroviral vectors (pMXs) containing the murine complementary DNAs of Oct4, Sox2, and Klf4¹ were obtained from Addgene. These plasmids were co-transfected into 293T cells with packaging vectors (pUMVC and pCMV-VSVG), and viral supernatants were collected 48 hours post-transfection to infect human fibroblasts. Two to four rounds of infection were performed during a 48 hour period. The typical infection efficiency is 70-90%, judging by expression of a control GFP vector or immunofluorescence staining of Oct4 or Sox2. The day that viral supernatants were removed was defined as 0 day post-infection.

Induction of iPS Cells

Human fibroblasts were infected by different transcription factor combinations, and replated in fibroblast medium typically at 2×10⁵ cells per well in gelatin-coated 6-well plates at 0 day post-infection. Cells were cultured in hES cell media starting from 1 day post-infection. Treatment with VPA (0.5-1 mM) begins typically at 1 days post-infection, and lasts for up to 2 weeks. iPS colonies were picked about 1 month post-infection based on colony morphology. The picked colonies were subsequently expanded and maintained on irradiated MEF feeder layers in hES cell media without VPA. Y-27632, a ROCK inhibitor that enhances survival of single dissociated hES and iPS cells^{7,21}, were used at 5-10 uM to increase the seeding efficiency of iPS cells for the initial colony expansion after picking and for the first two days after passaging. 3-factor (Oct4, Sox2 and Klf4) induced iPS cells from BJ friboblasts were named as “B 124-” followed by a number to distinguish between different clones Similarly, 2-factor (Oct4 and Sox2) induced iPS lines from BJ and NHDF fibroblasts were named as “B 12-” and “F12-” respectively followed by a number.

VPA and Y-27632 were purchased from EMD Biosciences, and stock solutions were made in media. Karyotyping of the iPS cell lines was performed by the Clinical & Research Cytogenetics Laboratories at the Oregon Health & Sciences University. DNA fingerprinting analysis was performed by CellLine Genetics.

Methods

In vitro Differentiation of Human iPS Cells

For spontaneous differentiation through embryoid body (EB) formation, human iPS cells were dissociated by collagenase IV treatment, and transferred to low attachment 6-well plates in Knockout DMEM supplemented with 20% Knockout serum replacement, non-essential amino acid, β-mercaptoethanol, L-glutamine and penicillin/streptomycin. After 8 days in suspension culture, EBs were transferred to gelatin-coated plates and cultured in the same medium for another 8 days.

Established protocols for directed differentiation of hES cells were used for the differentiation of human iPS cells into putative dopaminergic neurons and cardiomyocytes^{11,12}. For induction of definitive endoderm cells, an established protocol for hES cells using activin A treatment¹³ was employed. Briefly, undifferentiated human iPS cells at approximately 80% confluence were induced to differentiate into definitive endoderm cells with 100 ng/ml recombinant activin A (R&D) in RPMI1640 medium supplemented with 2% FCS, L-glutamine and penicillin/streptomycin for 4 days. For the induction of pancreatic progenitor cells, activin A-treated cells were cultured for 8 additional days in DMEM/F12 supplemented with N2 and B27 supplements, non-essential amino acids, β-mercaptoethanol, 0.5 mg/ml bovine serum albumin, L-glutamine and penicillin/streptomycin.

Teratoma Formation of Human iPS Cells

Human iPS cells grown on MEF feeder layers were collected by collagenase IV treatment, and injected subcutaneously into NOD-SCID mice. Palpable tumors were observed typically 1-2 month after injection. Tumor samples were collected in 2-3 months, and processed for paraffin embedding and hematoxylin and eosin staining following standard procedures.

Alkaline Phosphatase and Immunofluorescence Staining

AP staining was performed with the Vector Red substrate kit from Vector Laboratories Immunofluorescence staining was performed using the following primary antibodies: AFP (A8452, Sigma), cTNT (MS-295-P1, NeoMarkers), DESMIN (RB-9014, Lab Vision), GFAP (Z0334, DAKO), NANOG (AF1997, R&D Systems), NKX2.5 (sc-14033, Santa Cruz Biotechnology), OCT4 (sc-5279, Santa Cruz Biotechnology), PDX1 (AF2419, R&D systems), SMA (A5228, Sigma), SSEA4 (MAB4304, Chemicon), SOX2 (sc-17320, Santa Cruz Biotechnology), SOX17 (AF1924, R&D systems), TH (AB152, Chemicon), TRA-1-60 (MAB4360, Chemicon), TRA-1-81 (MAB4381, Chemicon), TUJ-1 (MMS-435P, Covance Research Products).

Whole-Genome Expression Analysis

For transcriptional analysis, total RNA was isolated from cells cultured in 6 well dishes using RNeasy Mini Kit and QlAshredder from Qiagen. Biotinylated antisense RNA were amplified using Illumina Total Prep RNA amplification Kit from Ambion, hybridized to Illumina Whole-Genome Expression BeadChips (HumanRef-8) and analyzed by Illumina Beadstation 500. All samples were prepared in two to three biological repeats. Data were analyzed using the Beadstudio software provided by Illumina.

Bisulphite Genomic Sequencing

Genomic DNA (1 μg) from all cell lines were processed simultaneously for bisulphite modification using CpGenome Universal DNA Modification Kit (Chemicon). The promoter regions of OCT4 were amplified by PCR using primer sets previously described^{7,22,23}. Primer sequences were provided in supplementary table 4. The PCR products were cloned into pCRII-TOPO vector using TOPO TA cloning kit (Invitrogen) and sequenced.

RT-PCR and PCR

Total RNA was isolated using RNeasy kit (Qiagen) followed by cDNA synthesis using Superscript III Reverse Transcriptase and Oligo (dT)12-18 primers (Invitrogen). PCR was performed with JumpStart Taq DNA polymerase (Sigma). qPCR was performed using QuantiFast SYBR Green PCR Kit (Qiagen) and analyzed with MJ_Opticon. Primer sequences were supplied in Table 5.

TABLE 5
primers for RT-PCR and PCR reactions.

| | | |
|---|---|---|
| Gene name | Forward primer (5′-to-3′) | Reverse primer (5′-to-3′) |

For RT-PCR

| | | |
|---|---|---|
| Oct4 (transgene) | GTGGTGGTACGGGAAATCAC | TAGCCAGGTTCGAGAATCCA |
| Sox2 (transgene) | GCATCGCAGCTTGGATACAC | GCTTCAGCTCCGTCTCCAT |
| 4-Oct | CTGGGTTGATCCTCGGACCT | CACAGAACTCATACGGCGGG |
| NANOG | AAAGAATCTTCACCTATGCC | GAAGGAAGAGGAGAGACAGT |
| TDGF | TCCTTCTACGGACGGAACTG | AGAAATGCCTGAGGAAAG CA |
| SOX1 | CACAACTCGGAGATCAGCAA | GGTACTTGTAATCCGGGTGC |
| PAX6 | GTCCATCTTTGCTTGGGAAA | TAGCCAGGTTGCGAAGAACT |
| MAP2 | CAGGTGGCGGACGTGTGAAAATTGAGAGTG | CACGCTGGATCTGCCTGGGGACTGT |
| BRACHYURY | AATTGGTCCAGCCTTGGAAT | CGTTGCTCACAGACCACA |
| FLK1 | TGATCGGAAATGACACTGGA | CACGACTCCATGTTGGTCAC |
| MSX1 | CGAGAGGACCCCGTGGATGCAGAG | GGCGGCCATCTTCAGCTTCTCCAG |
| SOX17 | CTCTGCCTCCTCCACGAA | CAGAATCCAGACCTGCACAA |
| HNF3β | GGAGCGGTGAAGATGGAA | TACGTGTTCATGCCGTTCAT |
| AFP | AAATGCGTTTCTCGTTGCTT | GCCACAGGCCAATAGTTTGT |
| AADC | CGCCAGGATCCCCGCTTTGAAATCTG | TCGGCCGCCAGCTCTTTGATGTGTTC |
| DAT | ACAGAGGGGAGGTGCGCCAGTTCACG | ACGGGGTGGACCTCGCTGCACAGAT |
| ChAT | GGAGGCGTGGAGCTCAGCGACACC | CGGGGAGCTCGCTGACGGAGTCTG |
| LMX1β | GGCACCAGCAGCAGCAGGAGCAGCAG | CCACGTCTGAGGAGCCGAGGAAGCA |
| TH | CTGTGGCCTTTGAGGAGAAG | ATGGTGGATTTTGGCTTCAA |
| NKX2.5 | CTCCCAACATGACCCTGAGT | CTCATTGCACGCTGCATAAT |
| cTNT | CAGAGCGGAAAAGTGGGAAGA | TCGTTGATCCTGTTTCGGAGA |
| MEF2C | TTTAACACCGCCAGCGCTCTTCACCTTG | TCGTGGCGCGTGTGTTGTGGGTATCT |
| MYL2A | GGGCCCCATCAACTTCACCGTCTTCC | TGTAGTCGATGTTCCCCGCCAGGTCC |
| MYHCB | CTGGAGGCCGAGCAGAAGCGCAACG | GTCCGCCCGCTCCTCTGCCTCATCC |
| HNF4α | CGAGCAGATCCAGTTCATCA | CGTTGGTTCCCATATGTTCC |
| PDX1 | CCTTTCCCATGGATGAAGTC | GGAACTCCTTCTCCAGCTCTA |
| GAPDH | GTGGACCTGACCTGCCGTCT | GGAGGAGTGGGTGTCGCTGT |
| β-ACTIN | CAATGTGGCCGAGGACTTTG | CATTCTCCTTAGAGAGAAGTGG |

For PCR to detect integration of viral transgenes

| | | |
|---|---|---|
| 4-Oct | GCATCGCAGCTTGGATACAC | CCAAGGTGATCCTCTTCTGC |
| Sox2 | GCATCGCAGCTTGGATACAC | GCTTCAGCTCCGTCTCCAT |
| Klf4 | GCATCGCAGCTTGGATACAC | AAGGCCCTGTCACACTTCTG |

For bisulphite-sequencing PCR

| | | |
|---|---|---|
| OCT4 (1) | GGATGTTATTAAGATGAAGATAGTTGG | CCTAAACTCCCCTTCAAAATCTATT |
| OCT4 (2) | TAGTTGGGATGTGTAGAGTTTGAGA | TAAACCAAAACAATCCTTCTACTCC |

### Reference (for Example 2):

¹ Takahashi, K. & Yamanaka, S. Induction of pluripotent stem cells from mouse embryonic and adult fibroblast cultures by defined factors. Cell 126 (4), 663-676 (2006).
² Okita, K., Ichisaka, T., & Yamanaka, S. Generation of germline-competent induced pluripotent stem cells. Nature 448 (7151), 313-317 (2007).
³ Maherali, N. et al. Directly Reprogrammed Fibroblasts Show Global Epigenetic Remodeling and Widespread Tissue Contribution. Cell Stem Cell 1 (1), 55-70 (2007).
⁴ Wernig, M. et al. In vitro reprogramming of fibroblasts into a pluripotent ES-cell-like state. Nature 448 (7151), 318-324 (2007).
⁵ Takahashi, K. et al. Induction of pluripotent stem cells from adult human fibroblasts by defined factors. Cell 131 (5), 861-872 (2007).
⁶ Yu, J. et al. Induced pluripotent stem cell lines derived from human somatic cells. Science 318 (5858), 1917-1920 (2007).
⁷ Park, I. H. et al. Reprogramming of human somatic cells to pluripotency with defined factors. Nature 451 (7175), 141-146 (2008).
⁸ Lowry, W. E. et al. Generation of human induced pluripotent stem cells from dermal fibroblasts. Proc Natl Acad Sci USA 105 (8), 2883-2888 (2008).
⁹ Nakagawa, M. et al. Generation of induced pluripotent stem cells without Myc from mouse and human fibroblasts. Nat Biotechnol 26 (1), 101-106 (2008).
¹⁰ Wernig, M., Meissner, A., Cassady, J. P., & Jaenisch, R. c-Myc is dispensable for direct reprogramming of mouse fibroblasts. Cell Stem Cell 2 (1), 10-12 (2008).
¹¹ Kawasaki, H. et al. Induction of midbrain dopaminergic neurons from ES cells by stromal cell-derived inducing activity. Neuron 28 (1), 31-40 (2000).
¹² Osafune, K. et al. Marked differences in differentiation propensity among human embryonic stem cell lines. Nat Biotechnol 26 (3), 313-315 (2008).
¹³ D'Amour, K. A. et al. Efficient differentiation of human embryonic stem cells to definitive endoderm. Nat Biotechnol 23 (12), 1534-1541 (2005).
¹⁴Yasunaga, M. et al. Induction and monitoring of definitive and visceral endoderm differentiation of mouse ES cells. Nat Biotechnol 23 (12), 1542-1550 (2005).
¹⁵ Kubo, A. et al. Development of definitive endoderm from embryonic stem cells in culture. Development 131 (7), 1651-1662 (2004).
¹⁶ Cowan, C. A. et al. Derivation of embryonic stem-cell lines from human blastocysts. N Engl J Med 350 (13), 1353-1356 (2004).
¹⁷ Rossant, J. Stem cells: the magic brew. Nature 448 (7151), 260-262 (2007).
¹⁸ Zaehres, H. & Scholer, H. R. Induction of pluripotency: from mouse to human Cell 131 (5), 834-835 (2007).
¹⁹ Perry, A. C. Induced pluripotency and cellular alchemy. Nat Biotechnol 24 (11), 1363-1364 (2006).
²⁰ Boyer, L. A. et al. Core transcriptional regulatory circuitry in human embryonic stem cells. Cell 122 (6), 947-956 (2005).
²¹ Watanabe, K. et al. A ROCK inhibitor permits survival of dissociated human embryonic stem cells. Nat. Biotechnol. 25 (6), 681-686 (2007).
²² Deb-Rinker, P. et al. Sequential DNA methylation of the Nanog and Oct-4 upstream regions in human NT2 cells during neuronal differentiation. J Biol Chem 280 (8), 6257-6260 (2005).
²³ Freberg, C. T., Dahl, J. A., Timoskainen, S., & Collas, P. Epigenetic reprogramming of OCT4 and NANOG regulatory regions by embryonal carcinoma cell extract. Mol Biol Cell 18 (5), 1543-1553 (2007).

### Supplementary Reference (for Example 2):

S1 Kawasaki, H. et al. Induction of midbrain dopaminergic neurons from ES cells by stromal cell-derived inducing activity. Neuron 28 (1), 31-40 (2000).
S2 Osafune, K. et al. Marked differences in differentiation propensity among human embryonic stem cell lines. Nat Biotechnol 26 (3), 313-315 (2008).
S3 D'Amour, K. A. et al. Efficient differentiation of human embryonic stem cells to definitive endoderm. Nat Biotechnol 23 (12), 1534-1541 (2005).
S4 Cowan, C. A. et al. Derivation of embryonic stem-cell lines from human blastocysts. N Engl J Med 350 (13), 1353-1356 (2004).

Other embodiments are within the following claims:

1. A method of producing an iPS cell from a somatic cell, the method comprising: treating the somatic cell with at least two transcription factors and contacting the somatic cell with a DNA methyl transferase inhibitor or a histone deacetylase (HDAC) inhibitor under conditions sufficient to produce an iPS cell from the somatic cell.

2. The method of claim 1, wherein the somatic cell is treated with at least two transcription factors prior to the step of contacting the somatic cell with the DNA methyl transferase inhibitor or the HDAC inhibitor.

3. (canceled)

4. (canceled)

5. The method of claim 1, wherein the DNA methyl transferase inhibitor comprises 5′-azacytidine.

6. The method of claim 1, wherein the HDAC inhibitor selectively inhibits a Class I or Class II HDAC.

7. The method of claim 6, wherein the HDAC inhibitor comprises VPA, SAHA or TSA, or a combination thereof.

8. (canceled)

9. The method of claim 1, wherein the method further comprises the step of contacting the cell with a glucocorticoid compound.

10. The method of claim 9, wherein the glucocorticoid compound comprises dexamethasone.

11. The method of claim 1, wherein the transcription factors comprise Oct4, Klf4, Sox2 or c-Myc.

12.-17. (canceled)

18. The method of claim 1, wherein the expression of a marker selected from a group consisting of alkaline phophatase, NANOG, OCT4, SOX2, SSEA4, TRA-1-60 and TRA-1-81, is upregulated to by a statistically significant amount in the iPS cell relative to the somatic cell.

19. (canceled)

20. The method of claim 1, wherein the somatic cell is a fibroblast, a muscle cell, a cumulus cell, a neural cell, a liver cell, a GI tract cell, a mammary cell, a hepatocyte or a pancreatic islet cell.

21. (canceled)

22. The method of claim 1, wherein the somatic cell is a human cell.

23. (canceled)

24. (canceled)

25. The method of claim 24, wherein the method further comprises isolating a population of the iPS cells.

26. (canceled)

27. The method of claim 25, wherein the method further comprises implanting the iPS cells in to a subject.

28. The method of claim 27, wherein the subject is suffering from a disorder.

29. The method of claim 28, wherein the iPS cells are from a donor different than the subject.

30.-42. (canceled)

43. A reaction mixture comprising a more primitive precursor or a less differentiated cell compared to a somatic cell from which it was derived, and an exogenously produced DNA methyl transferase inhibitor or HDAC inhibitor, or a combination thereof.

44.-51. (canceled)

52. A kit comprising: a somatic cell; at least one compound selected from a DNA methyl transferase inhibitor or an HDAC inhibitor, or a combination thereof; at least two transcription factors selected from the group consisting of Oct4, Sox2, Klf4 and c-Myc; and instructions for producing an iPS cell from a somatic cell.

53. The kit of claim 52, wherein the HDAC inhibitor comprises VPA.

54. The kit of claim 52, wherein the somatic cell is a human somatic cell.

55.-58. (canceled)

59. The kit of claim 52, wherein the kit further comprises: a component for the detection of a marker for an iPS cell selected from a group selected from a group consisting of alkaline phophatase, NANOG, OCT4, SOX2, SSEA4, TRA-1-60 and TRA-1-81.

60.-83. (canceled)
